(19) United States
(12) Patent Application Publication
(10) Pub. No.: US 2023/0309603 A1
(43) Pub. Date: Oct. 5, 2023

(54) AGGLOMERATED BOTANICAL MATERIAL FOR ORAL PRODUCTS

(71) Applicant: R.J. Reynolds Tobacco Company, Winston-Salem, NC (US)

(72) Inventor: Frank Kelley St. Charles, Bowling Green, KY (US)

(21) Appl. No.: 17/710,076

(22) Filed: Mar. 31, 2022

(51) Int. Cl. A24B 13/00; A61K 9/00; A61K 9/16; A61K 36/81; A61K 31/465; A61K 45/06; A24B 15/28; A24B 15/14; A24B 15/40; A24B 15/32; A24B 15/30

(52) U.S. Cl. CPC A24B 13/00; A61K 9/009; A61K 9/1635; A61K 9/1652; A61K 36/81; A61K 31/465; A61K 45/06; A24B 15/285; A24B 15/14; A24B 15/403; A24B 15/32; A24B 15/302

(57) ABSTRACT

The disclosure provides methods of preparing agglomerated botanical materials and associated oral products including such agglomerated botanical materials. Some aspects of the disclosure provide methods for preparing an agglomerated botanical material, which include combining botanical particle fines having an average particle size of about 300 microns or less with a binding agent, forming agglomerated botanical particles from the combined botanical particle fines and binding agent, and optionally, treating the agglomerated botanical particles to increase water stability.

### FIELD OF THE DISCLOSURE

The present disclosure relates to methods of preparing agglomerated botanical materials and oral products including such agglomerated botanical materials, prepared according to the methods described herein. The products are generally configured for oral use and deliver substances such as flavors and/or active ingredients during use. Such products may include tobacco or a product derived from tobacco, or may be tobacco-free alternatives.

### BACKGROUND

Tobacco may be enjoyed in a so-called “smokeless” form. Particularly popular smokeless tobacco products are employed by inserting some form of processed tobacco or tobacco-containing formulation into the mouth of the user. Conventional formats for such smokeless tobacco products include moist snuff, snus, and chewing tobacco, which are typically formed almost entirely of particulate, granular, or shredded tobacco, and which are either portioned by the user or presented to the user in individual portions, such as in single-use pouches or sachets. Other traditional forms of smokeless products include compressed or agglomerated forms, such as plugs, tablets, or pellets. Alternative product formats, such as tobacco-containing gums and mixtures of tobacco with other plant materials, are also known. See for example, the types of smokeless tobacco formulations, ingredients, and processing methodologies set forth in U.S. Pat. No. 1,376,586 to Schwartz; U.S. Pat. No. 4,513,756 to Pittman et al.; U.S. Pat. No. 4,528,993 to Sensabaugh, Jr. et al.; 4,624,269 to Story et al.; U.S. Pat. No. 4,991,599 to Tibbetts; U.S. Pat. No. 4,987,907 to Townsend; U.S. Pat. No. 5,092,352 to Sprinkle, III et al.; U.S. Pat. No. 5,387,416 to White et al.; U.S. Pat. No. 6,668,839 to Williams; U.S. Pat. No. 6,834,654 to Williams; U.S. Pat. No. 6,953,040 to Atchley et al.; U.S. Pat. No. 7,032,601 to Atchley et al.; and U.S. Pat. No. 7,694,686 to Atchley et al.; US Pat. Pub. Nos. 2004/0020503 to Williams; 2005/0115580 to Quinter et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0173317 to Robinson et al.; 2008/0209586 to Neilsen et al.; 2009/0065013 to Essen et al.; and 2010/0282267 to Atchley, as well as WO2004/095959 to Arnarp et al., each of which is incorporated herein by reference.

Typically, smokeless tobacco compositions known in the art include tobacco materials and in some cases, reconstituted tobacco materials, which serve as the base for the oral product compositions. During conventional tobacco processing operations, plant parts or pieces are often comminuted, ground or pulverized into a particulate form using equipment and techniques for grinding, milling, or the like. Examples of processes for producing reconstituted tobacco materials are known in the art. See, for example, U.S. Pat. No. 3,398,754 to Tughan; U.S. Pat. No. 3,847,164 to Mattina; U.S. Pat. No. 4,131,117 to Kite; U.S. Pat. No. 4,270,552 to Jenkins; U.S. Pat. No. 4,308,877 to Mattina; U.S. Pat. No. 4,341,228 to Keritsis; U.S. Pat. No. 4,421,126 to Gellatly; U.S. Pat. No. 4,706,692 to Gellatly; U.S. Pat. No. 4,962,774 to Thomasson; U.S. Pat. No. 4,941,484 to Clapp; U.S. Pat. No. 4,987,906 to Young; U.S. Pat. No. 5,056,537 to Brown; U.S. Pat. No. 5,143,097 to Sohn; U.S. Pat. No. 5,159,942 to Brinkley et al.; U.S. Pat. No. 5,325,877 to Young; U.S. Pat. No. 5,445,169 to Brinkley; U.S. Pat. No. 5,501,237 to Young; and U.S. Pat. No. 5,533,530 to Young, which are incorporated herein by reference.

While traditional tobacco processing operations have been around for years, such processes generally suffer from drawbacks due to particulate tobacco fines that are generated during operations. Such particulate tobacco fines can cause issues with machine operations, dusting, and yield loss to name a few. In particular, particulate tobacco fines are commonly referred to as tobacco waste and are discarded during the process as they have particle sizes that are much too small and are not useful for addition to typical tobacco blends used in oral products and aerosol provision systems. Aerosol provision systems include, for example, vapor products commonly known as “electronic cigarettes,” “e-cigarettes” or electronic nicotine delivery systems (ENDS), as well as heat-not-burn products including tobacco heating products (THPs) and carbon-tipped tobacco heating products (CTHPs).

### BRIEF SUMMARY

The present disclosure generally provides methods of preparing agglomerated botanical materials and products adapted for oral use including such agglomerated botanical materials. In some embodiments, the agglomerated botanical materials may optionally be treated to increase the stability thereof when in contact with water (e.g., having an improved wet strength). Although applicable to other botanical materials, the method of the disclosure is advantageous for use in agglomerating tobacco fines that may result from various tobacco product manufacturing processes. In certain embodiments, the method of the disclosure enables the beneficial use of tobacco or other botanical fines in various useful products, including products adapted for oral use, even such products that have a relatively high moisture content.

One aspect of the present disclosure provides a method of preparing an agglomerated botanical material having an increased water stability. In some embodiments, such methods may include combining botanical particle fines having an average particle size of about 300 microns or less with a binding agent, the binding agent being selected from the group consisting of alginates, chitosan, water-insoluble polymers, plant oils having a melting point above room temperature, and combinations thereof. Further, in some embodiments, such methods may comprise forming agglomerated botanical particles from the combined botanical particle fines and binding agent, the agglomerated botanical particles having an average particle size greater than the tobacco particle fines. In still further aspects of the disclosure, such methods may optionally include treating the agglomerated botanical particles to increase the water stability thereof.

In some embodiments of the disclosed methods, the combining step may comprise combining the botanical particle fines with the binding agent at a weight ratio of botanical particle fines to binding agent of from about 0.5:1 to about 5:1. In certain embodiments, the combining step may comprise combining the botanical particle fines with the binding agent at a weight ratio of botanical particle fines to binding agent of from about 0.8:1 to about 3.25:1. In some embodiments, the combining step may comprise combining the botanical particle fines with the binding agent at a weight ratio of botanical particle fines to binding agent of about 3.25 or less. Generally, the average particle size of the agglomerated botanical particles may vary. In some embodiments, for example, the agglomerated botanical particles have an average particle size greater than about 350 microns, or in other embodiments an average particle size greater than about 400 microns, or in still other embodiments an average particle size greater than about 500 microns.

The binding agent used in the methods disclosed herein may vary. In some embodiments, for example, the binding agent is a water-insoluble polymer selected from group consisting of polyvinyl ester polymers and polymeric cellulose derivatives. In certain embodiments, the binding agent is a water-insoluble polymer selected from the group consisting of polyvinyl acetate and ethyl cellulose. In some aspects of the disclosure, the binding agent can be a water-insoluble polymer in the form of an aqueous emulsion, and the method can further comprise drying the agglomerated botanical particles. In still other embodiments, the binding agent can a plant oil having a melting point above room temperature, and the combining can optionally be conducted at elevated temperature. In such embodiments, the disclosed methods may further comprise cooling the agglomerated botanical particles to solidify the plant oil.

In certain aspects of the disclosure, the binding agent may be chitosan or an alginate salt, optionally combined with hydroxypropyl methyl cellulose. For example, in some embodiments, the binding agent is chitosan and the optional treating step may comprise treating the agglomerated botanical particles to increase the pH to about 7 or higher. In some embodiments of the disclosed methods, the binding agent is an alginate salt and the optional treating step may comprise treating the agglomerated botanical particles with a divalent cation. In such embodiments, the divalent cation can comprise calcium ions. In still further embodiments, the binding agent can be an alginate salt combined with hydroxypropyl methyl cellulose and the optional treating step may comprise treating the agglomerated botanical particles with a divalent cation solution at elevated temperature. In some aspects of the disclosed methods, the botanical particle fines may comprise stems of a plant of the Nicotiana species.

Another aspect of the present disclosure provides a product adapted for oral use. In such embodiments, the product adapted for oral use may comprise a plurality of agglomerated botanical particles, each agglomerated botanical particle comprising a plurality of botanical particle fines having an average particle size of about 300 microns and a binding agent. In certain embodiments, the binding agent can be selected from the group consisting of alginates, chitosan, water-insoluble polymers, plant oils having a melting point above room temperature, and combinations thereof. In some embodiments, the product may include an outer-water permeable pouch enclosing the agglomerated botanical particles.

In some aspects of the present disclosure, the oral products provided herein may comprise an active ingredient selected from the group consisting of a tobacco material, a nicotine component, additional botanicals, nutraceuticals, stimulants, amino acids, vitamins, cannabinoids, cannabimimetics, terpenes, and combinations thereof. In certain embodiments, the active ingredient is absorbed in or adsorbed on the agglomerated botanical particles. In other aspects, oral products according to the present disclosure may further comprise one or more additives selected from the group consisting of a flavoring agent, a salt, a sweetener, a filler, an additional binding agent, water, a humectant, an organic acid, a buffering agent, and combinations thereof. In such embodiments, the one or more additives can be absorbed in or adsorbed on the agglomerated botanical particles. In some embodiments, oral products according to the disclosure may have a water content in the range of about 20% to about 60% by weight, based on the total weight of the product. In other embodiments, oral products as provided herein may have a water content in the range of about 30% to about 50%, based on the total weight of the oral product.

The disclosure includes, without limitations, the following embodiments.

Embodiment 1: A method of preparing an agglomerated botanical material having an increased water stability, comprising: combining botanical particle fines having an average particle size of about 300 microns or less with a binding agent, the binding agent being selected from the group consisting of alginates, chitosan, water-insoluble polymers, plant oils having a melting point above room temperature, and combinations thereof forming agglomerated botanical particles from the combined botanical particle fines and binding agent, the agglomerated botanical particles having an average particle size greater than the tobacco particle fines; and optionally, treating the agglomerated botanical particles to increase the water stability thereof.

Embodiment 2: The method of claim 1, wherein the combining step comprises combining the botanical particle fines with the binding agent at a weight ratio of botanical particle fines to binding agent of from about 0.5:1 to about 5:1.

Embodiment 3: The method according to any one of embodiments 1-2, wherein the combining step comprises combining the botanical particle fines with the binding agent at a weight ratio of botanical particle fines to binding agent of from about 0.8:1 to about 3.25:1.

Embodiment 4: The method according to any one of embodiments 1-3, wherein the combining step comprises combining the botanical particle fines with the binding agent at a weight ratio of botanical particle fines to binding agent of about 3.25 or less.

Embodiment 5: The method according to any one of embodiments 1-4, wherein the agglomerated botanical particles have an average particle size greater than about 350 microns.

Embodiment 6: The method according to any one of embodiments 1-5, wherein the agglomerated botanical particles have an average particle size greater than about 400 microns.

Embodiment 7: The method according to any one of embodiments 1-6, wherein the agglomerated botanical particles have an average particle size greater than about 500 microns.

Embodiment 8: The method according to any one of embodiments 1-7, wherein the binding agent is a water-insoluble polymer selected from group consisting of polyvinyl ester polymers and polymeric cellulose derivatives.

Embodiment 9: The method according to any one of embodiments 1-8, wherein the binding agent is a water-insoluble polymer selected from the group consisting of polyvinyl acetate and ethyl cellulose.

Embodiment 10: The method according to any one of embodiments 1-9, wherein the binding agent is a water-insoluble polymer in the form of an aqueous emulsion, and the method further comprises drying the agglomerated botanical particles.

Embodiment 11: The method according to any one of embodiments 1-10, wherein the binding agent is a plant oil having a melting point above room temperature, and wherein the combining is optionally conducted at elevated temperature.

Embodiment 12: The method of embodiment 11, further comprises cooling the agglomerated botanical particles to solidify the plant oil.

Embodiment 13: The method according to any one of embodiments 1-12, wherein the binding agent is chitosan or an alginate salt, optionally combined with hydroxypropyl methyl cellulose.

Embodiment 14: The method according to any one of embodiments 1-13, wherein the binding agent is chitosan and the optional treating step comprises treating the agglomerated botanical particles to increase the pH to about 7 or higher.

Embodiment 15: The method according to any one of embodiments 1-14, wherein the binding agent is an alginate salt and the optional treating step comprises treating the agglomerated botanical particles with a divalent cation.

Embodiment 16: The method of embodiment 15, wherein the divalent cation comprises calcium ions.

Embodiment 17: The method according to any one of embodiments 1-16, wherein the binding agent is an alginate salt combined with hydroxypropyl methyl cellulose and the optional treating step comprises treating the agglomerated botanical particles with a divalent cation solution at elevated temperature.

Embodiment 18: The method according to any one of embodiments 1-17, wherein the botanical particle fines comprise stems of a plant of the Nicotiana species.

Embodiment 19: A product adapted for oral use comprising a plurality of agglomerated botanical particles, each agglomerated botanical particle comprising a plurality of botanical particle fines having an average particle size of about 300 microns and a binding agent, the binding agent being selected from the group consisting of alginates, chitosan, water-insoluble polymers, plant oils having a melting point above room temperature, and combinations thereof.

Embodiment 20: The product of embodiment 19, wherein the product comprises an outer-water permeable pouch enclosing the agglomerated botanical particles.

Embodiment 21: The product according to any one of embodiments 19-20, further comprising an active ingredient selected from the group consisting of a tobacco material, a nicotine component, additional botanicals, nutraceuticals, stimulants, amino acids, vitamins, cannabinoids, cannabimimetics, terpenes, and combinations thereof.

Embodiment 22: The product of embodiment 21, wherein the active ingredient is absorbed in or adsorbed on the agglomerated botanical particles.

Embodiment 23: The product according to any one of embodiments 19-22, further comprising one or more additives selected from the group consisting of a flavoring agent, a salt, a sweetener, a filler, an additional binding agent, water, a humectant, an organic acid, a buffering agent, and combinations thereof.

Embodiment 24: The product of embodiment 23, wherein the one or more additives is absorbed in or adsorbed on the agglomerated botanical particles.

Embodiment 25: The product according to any one of embodiments 19-24, wherein the product has a water content in the range of about 20% to about 60% by weight, based on the total weight of the product.

Embodiment 26: The product according to any one of embodiments 19-25, wherein the product has a water content in the range of about 30% to about 50%, based on the total weight of the oral product.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWING

Having thus described aspects of the disclosure in the foregoing general terms, reference will now be made to the accompanying drawing, which is not necessarily drawn to scale. The drawing is exemplary only, and should not be construed as limiting the disclosure.

FIG. 1 is a flow diagram illustrating the general steps of a method for producing reconstituted tobacco according to an embodiment of the present disclosure; and

FIG. 2 is a perspective view of a product adapted for oral use, wherein the product comprises an outer-water permeable pouch enclosing agglomerated botanical particles, according to an example embodiment of the present disclosure.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art.

Indeed, the disclosure may be embodied in many different forms and should not be construed as limited to the embodiments set forth herein; rather, these embodiments are provided so that this disclosure will satisfy applicable legal requirements. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water). Reference to “wet weight” refers to the weight of the mixture including water. Unless otherwise indicated, reference to “weight percent” of a mixture reflects the total wet weight of the mixture (i.e., including water).

As described herein, embodiments of the disclosure relate to methods of preparing an agglomerated botanical material having increased stability when in contact with water (e.g., having an improved wet strength). As noted above, during processing of tobacco materials, e.g., such as reconstituted tobacco materials and other botanical or plant-based materials, a considerable amount of botanical particle fines are generated during milling and subsequent processing. When preparing oral products or similar botanical-containing products, it is noted that these botanical particle fines add very little to the fill value of the tobacco or botanical blend since these fines fill in the void space between larger particles retained during processing. Although tobacco fines may be incorporated into known reconstituted tobacco processes, such processes produce materials that generally exhibit poor water stability, meaning the reconstituted tobacco materials are unsuitable for use in high moisture environments, such as in certain classes of oral tobacco products. In contrast, the present disclosure provides methods of processing tobacco fines that allow for recovery and reuse of such materials in a variety of product formats, including oral products exhibiting high moisture levels.

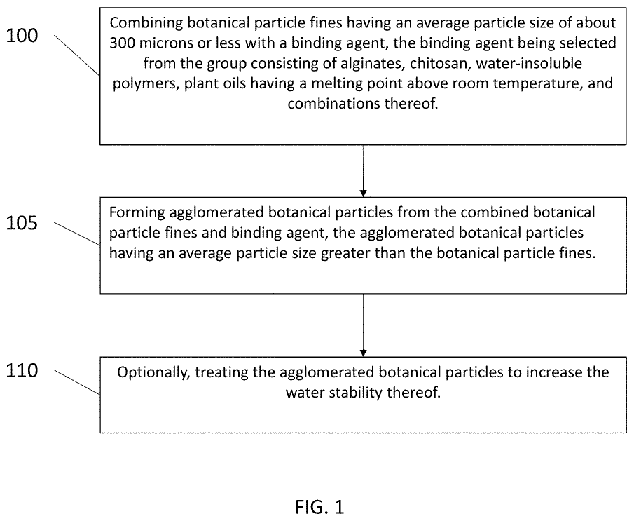

In one aspect, the present disclosure provides a method of preparing an agglomerated botanical material having an increased water stability/wet strength. As used herein, the terms “water stability” and “wet strength” are used interchangeably and generally refer to the ability of the agglomerated botanical particles to resist breakdown when contacted by water or other source of moisture, e.g., such as saliva. For example, an agglomerated botanical material having an increased water stability would be more resistant to breakdown/dissolution in water than a comparable botanical material that has not been treated according to the methods of the present disclosure. As illustrated in FIG. 1, for example, such methods comprise combining botanical particle fines having an average particle size of about 300 microns or less with a binding agent as shown at operation 100. “Botanical particles fines” as used herein, generally refers to any botanical material (e.g., including a tobacco material or other non-tobacco plant-based material) having an average particle size of 300 microns or less (or 250 microns or less, or 200 microns or less), such as materials having an average particle size range of about 10 to about 300 microns or about 50 to about 300 microns or about 100 microns to about 300 microns. As used herein, the terms “particulate” or “particles” generally refer to a material in the form of a plurality of individual particles, some of which can be in the form of an agglomerate of multiple particles. In various embodiments, the particles of a particulate material can be described as substantially spherical or granular.

The particle size of a particulate material may be measured by sieve analysis. As the skilled person will readily appreciate, sieve analysis (otherwise known as a gradation test) is a method used to measure the particle size distribution of a particulate material. Typically, sieve analysis involves a nested column of sieves which comprise screens, preferably in the form of wire mesh cloths. A pre-weighed sample may be introduced into the top or uppermost sieve in the column, which has the largest screen openings or mesh size (i.e. the largest pore diameter of the sieve). Each lower sieve in the column has progressively smaller screen openings or mesh sizes than the sieve above. Typically, at the base of the column of sieves is a receiver portion to collect any particles having a particle size smaller than the screen opening size or mesh size of the bottom or lowermost sieve in the column (which has the smallest screen opening or mesh size).

Generally, the types of botanical fines used according to the present disclosure may vary. For example, botanical particle fines may be recovered from any typical tobacco or non-tobacco plant material commonly used in the preparation of smokeless tobacco products and other oral product compositions. Example botanical materials are set forth herein below. While the following description refers to botanical particle fines derived from tobacco materials, it is noted that use of tobacco-derived fines is not intended to be limiting and the methods of the present disclosure provide for agglomeration of particulate fines derived from any plant-based source, including non-tobacco source for example. In addition, in some embodiments, the agglomerated particle fines may be combined with one or more larger tobacco particles and/or non-tobacco, plant-based particles to prepare oral products as described herein.

In some embodiments, the botanical particle fines can be derived from one or more components from a plant of the Nicotiana species including leaves, seeds, flowers, stalks, roots, and/or stems. Methods of the present disclosure can comprise harvesting a plant from the Nicotiana species and, in certain embodiments, separating certain components from the plant such as the stalks and/or roots, and physically processing these components. Although the botanical particle fines may be derived from whole tobacco plants or any component thereof (e.g., leaves, flowers, stems, roots, stalks, and the like), it can be advantageous to use stems, stalks and/or roots of the tobacco plant. For example, tobacco stems are commonly used in making reconstituted tobacco materials. As used herein, the term “stem” refers to the part of the plant that supports leaves and flowers. The botanical particle fines may also comprise tobacco lamina, tobacco scrap, and tobacco fines, including any waste tobacco materials formed during any part of the typical tobacco reconstitution process.

Typically, botanical particle fines as described herein are provided in a form that can be described as particulate (i.e., shredded, ground, granulated, or powder form). The manner by which the botanical particle fines are recovered and/or provided in a finely divided or powder type of form may vary. For example, during conventional tobacco processing operations, plant parts or pieces are comminuted, ground or pulverized into a particulate form using equipment and techniques for grinding, milling, or the like. In some embodiments, a plant material can be provided in relatively dry form during grinding or milling, using equipment such as hammer mills, cutter heads, air control mills, or the like. For example, tobacco parts or pieces may be ground or milled when the moisture content thereof is less than about 15 weight percent or less than about 5 weight percent. Generally, the botanical particle fines are employed in the form of parts or pieces that have an average particle size of 300 microns or less. In some instances, the tobacco particles may be sized to pass through a screen mesh to obtain the particle size range required. In other embodiments, the tobacco particles may be sized to pass through multiple screens to obtain the desired particle size range. If desired, air classification equipment may be used to ensure that small sized tobacco particles of the desired sizes, or range of sizes, may be collected. If desired, differently sized pieces of granulated tobacco may be mixed together.

As noted above, the botanical particle fines can be combined with a binding agent at operation 100. Typically, the binding agent can be selected from the group consisting of alginates, chitosan, water-insoluble polymers, plant oils having a melting point above room temperature, and combinations thereof. The particular binding agent combined with the botanical particle fines may vary based on the types of fines and/or the desired solubility characteristics thereof.

Water-insoluble polymers are those polymers that are insoluble or practically insoluble in water, such as those polymers having a water solubility of no more than 1 gram in 10,000 ml of water at room temperature and pressure (25° C. and 1 atm). For example, in some embodiments, the binding agent is a water-insoluble polymer selected from group consisting of polyvinyl ester polymers and polymeric cellulose derivatives. In some embodiments, the binding agent is a water-insoluble polymer selected from the group consisting of polyvinyl acetate (PVA), ethyl cellulose, acrylic polymers, waxes, latex, starches, resins, alcohols, polylactic polyglycolic acids, zein, dibutyl sebacate (DBS), triethyl citrate (TEC), tributyl citrate (TBC), acetyltributyl citrate (ATBC), amd acetyltriethyl citrate (ATEC), and the like. Water-insoluble polymers can be used in the form of, for example, an aqueous emulsion (e.g., such as a PVA emulsion, ethyl cellulose emulsion, and the like). It should be noted that some water-insoluble polymers (e.g., such as ethyl cellulose) may be soluble in one or more organic solvents (e.g., including, but not limited to, methanol and ethanol). Accordingly, in some embodiments, the water-insoluble binding agent may be used with a suitable organic solvent and combined with the botanical particle fines followed by a drying step to evaporate the organic solvent.

In some embodiments, the binding agent is a water-soluble binder such as chitosan or an alginate salt, optionally combined with hydroxypropyl methyl cellulose, which can be treated in some manner to reduce water solubility. For example, chitosan is water soluble at acidic pH, but becomes insoluble at alkaline pH. Alginate salts with monovalent cations (e.g., ammonia or sodium) are generally water soluble, but can crosslink in the presence of a crosslinking agent, such as divalent cations (e.g., calcium ions), to form a water-insoluble gel. The presence of certain cellulose derivatives (e.g., methyl cellulose, hydroxyethyl methyl cellulose, hydroxypropyl methyl cellulose, and the like) can also contribute to rapid gelling of the agglomerate at higher temperatures. For example, it should be noted that such rapid gelling gives the agglomerated botanical material stability in hot water while the alginate is rendered insoluble by the addition of divalent cations.

In some embodiments, the binding agent is a plant oil having a melting point above room temperature (25° C. at 1 atm). Examples include lipids that typically comprise mostly triglycerides along with lesser amounts of free fatty acids and mono- or diglycerides. Example plant-derived fats are comprised primarily of saturated or unsaturated fatty acid chains (most of which are bound within triglyceride structures) having a carbon length of about 10 to about 26 carbon atoms, or about 14 to about 20 carbon atoms, or about 14 to about 18 carbon atoms. In some embodiments, the lipid comprises an oil and, in particular, a food grade oil. Such oils include, but are not limited to, vegetable oils, such as acai oil, almond oil, amaranth oil, apricot oil, apple seed oil, argan oil, avocado oil, babassu oil, beech nut oil, ben oil, bitter gourd oil, black seed oil, blackcurrant seed oil, borage seed oil, borneo tallow nut oil, bottle gourd oil, brazil nut oil, buffalo gourd oil, butternut squash seed oil, cape chestnut oil, canola oil, carob cashew oil, cocoa butter, cocklebur oil, coconut oil, corn oil, cothune oil, coriander seed oil, cottonseed oil, date seed oil, dika oil, egus seed oil, evening primrose oil, false flax oil, flaxseed oil, grape seed oil, grapefruit seed oil, hazelnut oil, hemp oil, kapok seed oil, kenaf seed oil, lallemantia oil, lemon oil, linseed oil, macadamia oil, mafura oil, marula oil, meadowfoam seed oil, mongongo nut oil, mustard oil, niger seed oil, nutmeg butter, okra seed oil, olive oil, orange oil, palm oil, papaya seed oil, peanut oil, pecan oil, perilla seed oil, persimmon seed oil, pequi oil, pili nut oil, pine nut oil, pistachio oil, pomegranate seed oil, poppyseed oil, pracaxi oil, prune kernel oil, pumpkin seed oil, quinoa oil, ramtil oil, rapeseed oil, rice bran oil, royle oil, sacha inchi oil, safflower oil, sapote oil, seje oil, sesame oil, shea butter, soybean oil, sunflower oil, taramira oil, tea seed oil, thistle oil, tigernut oil, tobacco seed oil, tomato seed oil, walnut oil, watermelon seed oil, wheat germ oil, and combinations thereof. In certain embodiments, the plant-derived fats of the present disclosure include palm oil, palm kernel oil, soybean oil, cottonseed oil, and mixtures thereof. In one embodiment, the lipid is a blend of palm oil and palm kernel oil. The lipid can be, for example, hydrogenated, partially hydrogenated, or non-hydrogenated. Example embodiments of lipids can be purchased under the brand names CEBES®, CISAO®, or CONFAO®, available from AarhusKarlshamn USA Inc.

The melting point of the lipid is typically about 29° C. or above, such as about 29° C. to about 49° C., or about 36° C. to about 45° C., or about 38° C. to about 41° C. One test for determining the melting point of lipids is the Mettler dropping point method (ASTM D3954-15, Standard Test Method for Dropping Point of Waxes, ASTM International, West Conshohocken, P A, 2015, www.astm.org.).

Generally, the amount of binding agent combined with the botanical particle fines may vary. In some embodiments, for example, the weight ratio of botanical particle fines to binding agent may be in the range of about 10:1 to about 0.5:1, about 8:1 to about 1:1, or about 5:1 to about 2:1. In some embodiments, the weight ratio of the botanical particle fines to the binding agent may be about 5:1 or less, about 4:1 or less, about 3:1 or less, about 2:1 or less, or about 1:1 or less, based on the total combined weight of the botanical particle fines and the binding agent. In certain embodiments, the weight ratio of the botanical particle fines to the binding agent may be in the range from about 0.5:1 to about 5:1, about 0.6:1 to about 4:1, about 0.7:1 to about 3.5:1, or about 0.8:1 to about 3.25:1.

The botanical fines and binding agent (and optionally other tobacco or plant-based particulates) may be contacted, combined, or mixed together using any mixing technique or equipment known in the art. Any mixing method that brings the composition ingredients into intimate contact can be used, such as a mixing apparatus featuring an impeller or other structure capable of agitation. Examples of mixing equipment include fluidized beds, casing drums, conditioning cylinders or drums, liquid spray apparatus, conical-type blenders, ribbon blenders, mixers available as FKM130, FKM600, FKM1200, FKM2000 and FKM3000 from Littleford Day, Inc., Plough Share types of mixer cylinders, Hobart mixers, and the like. See also, for example, the types of methodologies set forth in U.S. Pat. No. 4,148,325 to Solomon et al.; U.S. Pat. No. 6,510,855 to Korte et al.; and U.S. Pat. No. 6,834,654 to Williams, each of which is incorporated herein by reference.

As shown at operation 105, the methods of the present disclosure further comprise forming agglomerated botanical particles from the combined botanical particle fines and binding agent, for example, such that the agglomerated botanical particles have an average particle size greater than the botanical particle fines. The average particle size of the agglomerated botanical particles may vary. In some embodiments, for example, the agglomerated botanical particles can have an average particle size of greater than about 350 microns, greater than about 400 microns, greater than about 450 microns, greater than about 500 microns, or greater than about 600 microns. In various embodiments, the agglomerated botanical particles can have an average particle size in the range of about 350 microns to about 1000 microns, about 350 microns to about 750 microns, about 350 microns to about 600 microns, or about 350 microns to about 500 microns.

The step of forming agglomerated particles can include, for example, any suitable means for granulation. For example, granulation can be conducted in a granulator under high-shear, low-shear, fluid bed, rotor, or melt granulation techniques. Alternatively, the binding agent and botanical fines can be mixed to form a dough-like material and then pressed through a screen or sieve having openings of a predetermined size to form agglomerated particles. The resulting particles can be further treated with sieves or screens to further refine the average particle size as desired.

As noted herein, the agglomerated botanical particles can optionally be treated to increase the water stability thereof. For example, as demonstrated at operation 110 of FIG. 1, the agglomerated botanical particles can be treated to increase the water stability thereof. The types of treatments may vary based on the type of binding agent used. In some embodiments, for example, the binding agent is chitosan and the optional treating step comprises treating the agglomerated botanical particles to increase the pH to about 7 or higher by addition of a base. In some embodiments, the optional treating step may comprise treating the agglomerated botanical particles to increase the pH to at least 7, at least 8, at least 9, or at least 10. Any base can be used, such as strong bases (e.g., sodium hydroxide) or weak bases or buffering systems, such as carbonate salts and/or bicarbonate salts.

In other embodiments, the binding agent is an alginate salt and the optional treating step comprises treating the agglomerated botanical particles with divalent cations. In some embodiments, the divalent cations comprise calcium ions. In still other embodiments, the binding agent is an alginate salt combined with hydroxypropyl methyl cellulose and the optional treating step comprises treating the agglomerated botanical particles with a divalent cation at elevated temperature, such as a temperature of about 50° C. or higher, or about 60° C. or higher, or about 70° C. or higher (e.g., about 50° C. to about 95° C.).

In some aspects of the disclosure, the methods provided herein may further comprise one or more additional steps, e.g., such as drying the agglomerated tobacco material. In some embodiments, for example, the binding agent is a water-insoluble polymer in the form of an aqueous emulsion and the method further comprises drying the agglomerated particle fines. In some embodiments, for example wherein the binding agent is a plant oil, the method may further comprise cooling the agglomerated tobacco particles to room temperature in order to solidify the plant oil.

### Products Adapted for Oral Use

Some aspects of the present disclosure provide compositions and products adapted for oral use. The term “adapted for oral use” as used herein means that the product is provided in a form such that during use, saliva in the mouth of the user causes one or more of the components of the mixture (e.g., flavoring agents and/or nicotine) to pass into the mouth of the user. In certain embodiments, the product is adapted to deliver active ingredients and one or more additional components to a user through mucous membranes in the user's mouth and, in some instances, said active ingredient (including, but not limited to, for example, nicotine) can be absorbed through the mucous membranes in the mouth when the product is used.

Products adapted for oral use as described herein may take various forms, including moist snuff (e.g., such as American fermented snuff, heat-treated Swedish-style snuff, and the like), dry snuff, snus, powders, shredded or particulate form, and/or in the form of pouches. Certain products can exhibit, for example, one or more of the following characteristics: crispy, granular, chewy, syrupy, pasty, fluffy, smooth, and/or creamy. In certain embodiments, the desired textural property can be selected from the group consisting of adhesiveness, cohesiveness, density, dryness, fracturability, graininess, gumminess, hardness, heaviness, moisture absorption, moisture release, mouthcoating, roughness, slipperiness, smoothness, viscosity, wetness, and combinations thereof.

In one embodiment, products comprising the agglomerated botanical particles of the present disclosure may be in the form of a mixture disposed within a moisture-permeable container (e.g., a water-permeable pouch). Such mixtures in the water-permeable pouch format are typically used by placing one pouch containing the mixture in the mouth of a human subject/user. Generally, the pouch is placed somewhere in the oral cavity of the user, for example under the lips, in the same way as moist snuff products are generally used. The pouch preferably is not chewed or swallowed. Exposure to saliva then causes some of the components of the mixture therein (e.g., flavoring agents and/or active ingredients, such as nicotine or tobacco) to pass through e.g., the water-permeable pouch and provide the user with flavor and satisfaction, and the user is not required to spit out any portion of the mixture. After about 10 minutes to about 60 minutes, typically about 15 minutes to about 45 minutes, of use/enjoyment, substantial amounts of the mixture have been ingested by the human subject, and the pouch may be removed from the mouth of the human subject for disposal.

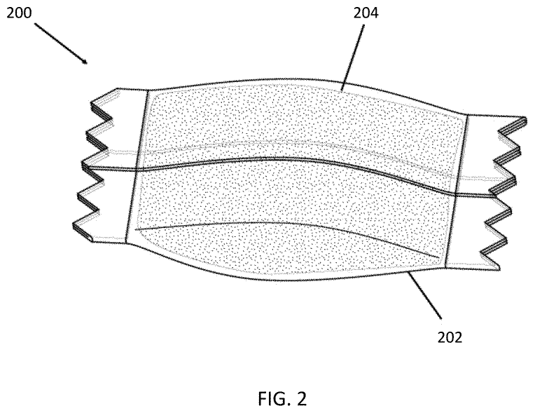

Accordingly, in certain embodiments, the mixture as disclosed herein (e.g., including the agglomerated botanical particles) and any other components noted herein below in more detail (e.g., such as flavoring agents, other active ingredients, other tobacco or plant-based particulates, and/or one or more additional additives) are combined within a moisture-permeable packet or pouch that acts as a container for use of the mixture to provide a pouched product configured for oral use. Certain embodiments of the disclosure will be described with reference to FIG. 2, and these described embodiments involve pouched or snus-type products having an outer pouch and containing a mixture as described herein. As explained in greater detail below, such embodiments are provided by way of example only, and the pouched products of the present disclosure can include the mixture of ingredients in other forms as desired. The mixture/construction of such packets or pouches, such as the container pouch 202 in the embodiment illustrated in FIG. 2, may be varied. Referring to FIG. 2, there is shown an embodiment of a pouched product 200. The pouched product 200 includes a moisture-permeable container in the form of a pouch 202, which contains a material 204 comprising at least the agglomerated botanical particles as described herein. The pouched product 200 may be an example of a product as described herein formed at least in part from the described compositions.

Suitable packets, pouches or containers of the type used for the manufacture of smokeless tobacco and oral products are available under the tradenames CatchDry, Ettan, General, Granit, Goteborgs Rape, Grovsnus White, Metropol Kaktus, Mocca Anis, Mocca Mint, Mocca Wintergreen, Kicks, Probe, Prince, Skruf and TreAnkrare. The mixture may be contained in pouches and packaged, in a manner and using the types of components used for the manufacture of conventional snus types of products. The pouch provides a liquid-permeable container of a type that may be considered to be similar in character to the mesh-like type of material that is used for the construction of a tea bag. Components of the mixture readily diffuse through the pouch and into the mouth of the user.

Non-limiting examples of suitable types of pouches are set forth in, for example, U.S. Pat. No. 5,167,244 to Kjerstad and U.S. Pat. No. 8,931,493 to Sebastian et al.; as well as US Patent App. Pub. Nos. 2016/0000140 to Sebastian et al.; 2016/0073689 to Sebastian et al.; 2016/0157515 to Chapman et al.; and 2016/0192703 to Sebastian et al., each of which are incorporated herein by reference. Pouches can be provided as individual pouches, or a plurality of pouches (e.g., 2, 4, 5, 10, 12, 15, 20, 25 or 30 pouches) can be connected or linked together (e.g., in an end-to-end manner) such that a single pouch or individual portion can be readily removed for use from a one-piece strand or matrix of pouches.

An example pouch may be manufactured from materials, and in such a manner, such that during use by the user, the pouch undergoes a controlled dispersion or dissolution. Such pouch materials may have the form of a mesh, screen, perforated paper, permeable fabric, or the like. For example, pouch material manufactured from a mesh-like form of rice paper, or perforated rice paper, may dissolve in the mouth of the user. As a result, the pouch and mixture each may undergo complete dispersion within the mouth of the user during normal conditions of use, and hence the pouch and mixture both may be ingested by the user. Other examples of pouch materials may be manufactured using water dispersible film forming materials (e.g., binding agents such as alginates, carboxymethylcellulose, xanthan gum, pullulan, and the like), as well as those materials in combination with materials such as ground cellulosics (e.g., fine particle size wood pulp). Preferred pouch materials, though water dispersible or dissolvable, may be designed and manufactured such that under conditions of normal use, a significant amount of the mixture contents permeate through the pouch material prior to the time that the pouch undergoes loss of its physical integrity. If desired, flavoring ingredients, disintegration aids, and other desired components, may be incorporated within, or applied to, the pouch material.

The amount of material contained within each product unit, for example, a pouch, may vary. In some embodiments, the weight of the mixture within each pouch is at least about 50 mg, for example, from about 50 mg to about 1 gram, from about 100 to 800 about mg, or from about 200 to about 700 mg. In some smaller embodiments, the weight of the mixture within each pouch may be from about 100 to about 300 mg. For a larger embodiment, the weight of the material within each pouch may be from about 300 mg to about 700 mg. If desired, other components can be contained within each pouch. For example, at least one flavored strip, piece or sheet of flavored water dispersible or water soluble material (e.g., a breath-freshening edible film type of material) may be disposed within each pouch along with or without at least one capsule. Such strips or sheets may be folded or crumpled in order to be readily incorporated within the pouch. See, for example, the types of materials and technologies set forth in U.S. Pat. No. 6,887,307 to Scott et al. and U.S. Pat. No. 6,923,981 to Leung et al.; and The EFSA Journal (2004) 85, 1-32; which are incorporated herein by reference.

A pouched product as described herein can be packaged within any suitable inner packaging material and/or outer container. See also, for example, the various types of containers for smokeless and oral types of products that are set forth in U.S. Pat. No. 7,014,039 to Henson et al.; U.S. Pat. No. 7,537,110 to Kutsch et al.; U.S. Pat. No. 7,584,843 to Kutsch et al.; U.S. Pat. No. 8,397,945 to Gelardi et al., D592,956 to Thiellier; D594,154 to Patel et al.; and D625,178 to Bailey et al.; US Pat. Pub. Nos. 2008/0173317 to Robinson et al.; 2009/0014343 to Clark et al.; 2009/0014450 to Bjorkholm; 2009/0250360 to Bellamah et al.; 2009/0266837 to Gelardi et al.; 2009/0223989 to Gelardi; 2009/0230003 to Thiellier; 2010/0084424 to Gelardi; and 2010/0133140 to Bailey et al; 2010/0264157 to Bailey et al.; and 2011/0168712 to Bailey et al. which are incorporated herein by reference.

Material within the Pouch

As noted above, some products prepared according to the present disclosure can comprise, in addition to the pouch-based exterior, a material within the pouch that typically comprises agglomerated botanical particles as described herein and, optionally, one or more active ingredients and/or one or more flavorants and/or other tobacco or plant-based particulates and/or various other optional ingredients. The composition of the material within the pouches provided herein is not particularly limited, and can comprise any additional filling composition known in the art (e.g., in addition to the agglomerated botanical particles, including those included within conventional pouched products. Such compositions are generally mixtures of two or more components and as such, the compositions are, in some cases, referenced herein below as “mixtures.” Certain components that can advantageously be included in the mixtures within certain embodiments of the pouched products provided herein are outlined generally below; however, it is to be understood that the discussion below is not intended to be limiting of the components that can be incorporated within the disclosed pouched products.

### Active Ingredient

The material or composition as disclosed herein may include one or more active ingredients. As used herein, an “active ingredient” refers to one or more substances belonging to any of the following categories: API (active pharmaceutical ingredient), food additives, natural medicaments, and naturally occurring substances that can have an effect on humans. Example active ingredients include any ingredient known to impact one or more biological functions within the body, such as ingredients that furnish pharmacological activity or other direct effect in the diagnosis, cure, mitigation, treatment, or prevention of disease, or which affect the structure or any function of the body of humans (e.g., provide a stimulating action on the central nervous system, have an energizing effect, an antipyretic or analgesic action, or an otherwise useful effect on the body). In some embodiments, the active ingredient may be of the type generally referred to as dietary supplements, nutraceuticals, “phytochemicals” or “functional foods.” These types of additives are sometimes defined in the art as encompassing substances typically available from naturally-occurring sources (e.g., botanical materials) that provide one or more advantageous biological effects (e.g., health promotion, disease prevention, or other medicinal properties), but are not classified or regulated as drugs.

Non-limiting examples of active ingredients include those falling in the categories of botanical ingredients, stimulants, amino acids, nicotine components, and/or pharmaceutical, nutraceutical, and medicinal ingredients (e.g., vitamins, such as A, B3, B6, B12, and C, and/or cannabinoids, such as tetrahydrocannabinol (THC) and cannabidiol (CBD)). Each of these categories is further described herein below. The particular choice of active ingredients will vary depending upon the desired flavor, texture, and desired characteristics of the particular product.

In certain embodiments, the active ingredient is selected from the group consisting of caffeine, taurine, GABA, theanine, vitamin C, lemon balm extract, ginseng, citicoline, sunflower lecithin, and combinations thereof. For example, the active ingredient can include a combination of caffeine, theanine, and optionally ginseng. In another embodiment, the active ingredient includes a combination of theanine, gamma-amino butyric acid (GABA), and lemon balm extract. In a further embodiment, the active ingredient includes theanine, theanine and tryptophan, or theanine and one or more B vitamins (e.g., vitamin B6 or B12). In a still further embodiment, the active ingredient includes a combination of caffeine, taurine, and vitamin C.

The particular percentages of active ingredients present will vary depending upon the desired characteristics of the particular product. Typically, an active ingredient or combination thereof is present in a total concentration of at least about 0.001% by weight of the material, such as in a range from about 0.001% to about 20%. In some embodiments, the active ingredient or combination of active ingredients is present in a concentration from about 0.1% w/w to about 10% by weight, such as, e.g., from about 0.5% w/w to about 10%, from about 1% to about 10%, from about 1% to about 5% by weight, based on the total weight of the material. In some embodiments, the active ingredient or combination of active ingredients is present in a concentration of from about 0.001%, about 0.01%, about 0.1%, or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the material. Further suitable ranges for specific active ingredients are provided herein below.

Botanical

In some embodiments, the active ingredient may comprise a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). In some embodiments, reference to botanical material may include tobacco, for example, as described herein above with regard to tobacco-derived botanical particle fines. However, in some embodiments, the botanical particle fines may not be derived from tobacco and may expressly exclude tobacco materials (i.e., does not include any Nicotiana species). For example, in some embodiments, the compositions as disclosed herein can be characterized as free of any tobacco material (e.g., any embodiment as disclosed herein may be completely or substantially free of any tobacco material). By “substantially free” is meant that no tobacco material has been intentionally added. For example, certain embodiments can be characterized as having less than 0.001% by weight of tobacco, or less than 0.0001%, or even 0% by weight of tobacco.

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the material.

The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include ashwagandha, Bacopa monniera, baobab, basil, Centella asiatica, Chai-hu, chamomile, cherry blossom, chlorophyll, cinnamon, citrus, cloves, cocoa, cordyceps, curcumin, damiana, Dorstenia arifolia, Dorstenia odorata, essential oils, eucalyptus, fennel, Galphimia glauca, ginger, Ginkgo biloba, ginseng (e.g., Panax ginseng), green tea, Griffonia simplicifolia, guarana, cannabis, hemp, hops, jasmine, Kaempferia parviflora (Thai ginseng), kava, lavender, lemon balm, lemongrass, licorice, lutein, maca, matcha, Nardostachys chinensis, oil-based extract of Viola odorata, peppermint, quercetin, resveratrol, Rhizoma gastrodiae, Rhodiola, rooibos, rose essential oil, rosemary, Sceletium tortuosum, Schisandra, Skullcap, spearmint extract, Spikenard, terpenes, tisanes, turmeric, Turnera aphrodisiaca, valerian, white mulberry, and Yerba mate.

In some embodiments, the active ingredient comprises lemon balm. Lemon balm (Melissa officinalis) is a mildly lemon-scented herb from the same family as mint (Lamiaceae). The herb is native to Europe, North Africa, and West Asia. The tea of lemon balm, as well as the essential oil and the extract, are used in traditional and alternative medicine. In some embodiments, the active ingredient comprises lemon balm extract. In some embodiments, the lemon balm extract is present in an amount of from about 1 to about 4% by weight, based on the total weight of the material.

In some embodiments, the active ingredient comprises ginseng. Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the ginseng is American ginseng or Korean ginseng. In specific embodiments, the active ingredient comprises Korean ginseng. In some embodiments, ginseng is present in an amount of from about 0.4 to about 0.6% by weight, based on the total weight of the material.

Stimulants

In some embodiments, the active ingredient comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis. In some embodiments, the active ingredient comprises caffeine. In some embodiments, the caffeine is present in an encapsulated form. On example of an encapsulated caffeine is Vitashure®, available from Balchem Corp., 52 Sunrise Park Road, New Hampton, NY, 10958.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the material. In some embodiments, the composition comprises caffeine in an amount of from about 1.5 to about 6% by weight, based on the total weight of the material;

Amino Acids

In some embodiments, the active ingredient comprises an amino acid. As used herein, the term “amino acid” refers to an organic compound that contains amine (—NH₂) and carboxyl (—COOH) or sulfonic acid (503H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-tranlational modification). Non-limiting examples of non-proteinogenic amino acids include gamma-aminobutyric acid (GABA), taurine (2-aminoethanesulfonic acid), theanine γ-glutamylethylamide), hydroxyproline, and beta-alanine. In some embodiments, the active ingredient comprises theanine. In some embodiments, the active ingredient comprises GABA. In some embodiments, the active ingredient comprises a combination of theanine and GABA. In some embodiments, the active ingredient is a combination of theanine, GABA, and lemon balm. In some embodiments, the active ingredient is a combination of caffeine, theanine, and ginseng. In some embodiments, the active ingredient comprises taurine. In some embodiments, the active ingredient is a combination of caffeine and taurine.

When present, an amino acid or combination of amino acids (e.g., theanine, GABA, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the material.

Vitamins

In some embodiments, the active ingredient comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). In some embodiments, the active ingredient comprises vitamin C. In some embodiments, the active ingredient is a combination of vitamin C, caffeine, and taurine.

When present, a vitamin or combination of vitamins (e.g., vitamin B6, vitamin B12, vitamin E, vitamin C, or a combination thereof) is typically at a concentration of from about 0.01% w/w to about 6% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, about 1%, about 2%, about 3%, about 4%, about 5%, or about 6% by weight, based on the total weight of the material.

Antioxidants

In some embodiments, the active ingredient comprises one or more antioxidants. As used herein, the term “antioxidant” refers to a substance which prevents or suppresses oxidation by terminating free radical reactions, and may delay or prevent some types of cellular damage. Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, Gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, Yerba mate, Yerba santa, Bacopa monniera, Withania somnifera, Lion's mane, and silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytoesterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12(2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the material.

Nicotine Component

In certain embodiments, the active ingredient comprises a nicotine component. By “nicotine component” is meant any suitable form of naturally-occurring or synthetic nicotine (e.g., free base or salt) for providing oral absorption of at least a portion of the nicotine present. Typically, the nicotine component is selected from the group consisting of nicotine free base and a nicotine salt. In some embodiments, the nicotine component is nicotine in its free base form, which easily can be adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine component can be employed in the form of a salt. Salts of nicotine can be provided using the types of ingredients and techniques set forth in U.S. Pat. No. 2,033,909 to Cox et al. and Perfetti, Beitrage Tabakforschung Int., 12: 43-54 (1983), which are incorporated herein by reference. Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride.

In some embodiments, at least a portion of the nicotine can be in the form of a resin complex of nicotine, where nicotine is bound in an ion-exchange resin, such as nicotine polacrilex, which is nicotine bound to, for example, a polymethacrilic acid, such as Amberlite IRP64, Purolite C115HMR, or Doshion P551. See, for example, U.S. Pat. No. 3,901,248 to Lichtneckert et al., which is incorporated herein by reference. Another example is a nicotine-polyacrylic carbomer complex, such as with Carbopol 974P. In some embodiments, nicotine may be present in the form of a nicotine polyacrylic complex.

Typically, the nicotine component (calculated as the free base) when present, is in a concentration of at least about 0.001% by weight of the material, such as in a range from about 0.001% to about 10%. In some embodiments, the nicotine component is present in a concentration from about 0.1% w/w to about 10% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the material. In some embodiments, the nicotine component is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the material.

In some embodiments, the products or compositions of the disclosure can be characterized as free of any nicotine component (e.g., any embodiment as disclosed herein may be completely or substantially free of any nicotine component). By “substantially free” is meant that no nicotine has been intentionally added, beyond trace amounts that may be naturally present in e.g., a botanical material. For example, certain embodiments can be characterized as having less than 0.001% by weight of nicotine, or less than 0.0001%, or even 0% by weight of nicotine, calculated as the free base.

In some embodiments, the active ingredient comprises a nicotine component (e.g., any product or composition of the disclosure, in addition to comprising any active ingredient or combination of active ingredients as disclosed herein, may further comprise a nicotine component).

Cannabinoids

In some embodiments, the active ingredient comprises one or more cannabinoids. As used herein, the term “cannabinoid” refers to a class of diverse chemical compounds that acts on cannabinoid receptors, also known as the endocannabinoid system, in cells that alter neurotransmitter release in the brain. Ligands for these receptor proteins include the endocannabinoids produced naturally in the body by animals; phytocannabinoids, found in cannabis; and synthetic cannabinoids, manufactured artificially. Cannabinoids found in cannabis include, without limitation: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), and tetrahydrocannabivarinic acid (THCV A). In certain embodiments, the cannabinoid is selected from tetrahydrocannabinol (THC), the primary psychoactive compound in cannabis, and cannabidiol (CBD) another major constituent of the plant, but which is devoid of psychoactivity. All of the above compounds can be used in the form of an isolate from plant material or synthetically derived.

Alternatively, the active ingredient can be a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids.

When present, a cannabinoid (e.g., CBD) or cannabimimetic is typically in a concentration of at least about 0.1% by weight of the material, such as in a range from about 0.1% to about 30%, such as, e.g., from about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 15%, about 20%, or about 30% by weight, based on the total weight of the material.

Terpenes

Active ingredients suitable for use in the present disclosure can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

Pharmaceutical Ingredients

In some embodiments, the active ingredient comprises an active pharmaceutical ingredient (API). The API can be any known agent adapted for therapeutic, prophylactic, or diagnostic use. These can include, for example, synthetic organic compounds, proteins and peptides, polysaccharides and other sugars, lipids, phospholipids, inorganic compounds (e.g., magnesium, selenium, zinc, nitrate), neurotransmitters or precursors thereof (e.g., serotonin, 5-hydroxytryptophan, oxitriptan, acetylcholine, dopamine, melatonin), and nucleic acid sequences, having therapeutic, prophylactic, or diagnostic activity. Non-limiting examples of APIs include analgesics and antipyretics (e.g., acetylsalicylic acid, acetaminophen, 3-(4-isobutylphenyl)propanoic acid), phosphatidylserine, myoinositol, docosahexaenoic acid (DHA, Omega-3), arachidonic acid (AA, Omega-6), S-adenosylmethionine (SAM), beta-hydroxy-beta-methylbutyrate (HMB), citicoline (cytidine-5′-diphosphate-choline), and cotinine. In some embodiments, the active ingredient comprises citicoline. In some embodiments, the active ingredient is a combination of citicoline, caffeine, theanine, and ginseng. In some embodiments, the active ingredient comprises sunflower lecithin. In some embodiments, the active ingredient is a combination of sunflower lecithin, caffeine, theanine, and ginseng.

The amount of API may vary. For example, when present, an API is typically at a concentration of from about 0.001% w/w to about 10% by weight, such as, e.g., from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the material.

In some embodiments, the composition is substantially free of any API. By “substantially free of any API” means that the composition does not contain, and specifically excludes, the presence of any API as defined herein, such as any Food and Drug Administration (FDA) approved therapeutic agent intended to treat any medical condition.

### Additional Components

In some embodiments, the material or composition may comprise one or more additional components, e.g., such as flavoring agents, binders, fillers, and the like. Non-limiting examples of additional components and inclusion rates thereof are provided herein below in more detail. It should be noted that the particular choice of additional components will vary depending upon the desired flavor, texture, and desired characteristics of the particular product.

Flavoring Agent

In some embodiments, the material or composition comprises a flavoring agent. As used herein, a “flavoring agent” or “flavorant” is any flavorful or aromatic substance capable of altering the sensory characteristics associated with the oral product. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy. In some embodiments, the material may include a single flavoring agent or a plurality of flavoring agents. If desired, one or more flavoring agents may be embedded within the fleece material, absorbed in or adsorbed on at least one surface of the fleece material, or impregnated within the fleece material.

Non-limiting examples of flavoring agents can include vanilla, coffee, chocolate/cocoa, cream, mint, spearmint, menthol, peppermint, wintergreen, eucalyptus, lavender, cardamon, nutmeg, cinnamon, clove, cascarilla, sandalwood, honey, jasmine, ginger, anise, sage, licorice, lemon, orange, apple, peach, lime, cherry, strawberry, terpenes, trigeminal sensates, and any combinations thereof. See also, Leffingwell et al., Tobacco Flavoring for Smoking Products, R. J. Reynolds Tobacco Company (1972), which is incorporated herein by reference. Flavorings also may include components that are considered moistening, cooling or smoothening agents, such as eucalyptus. These flavors may be provided neat (i.e., alone) or in a composite, and may be employed as concentrates or flavor packages (e.g., spearmint and menthol, orange and cinnamon; lime, pineapple, and the like). Representative types of components also are set forth in U.S. Pat. No. 5,387,416 to White et al.; US Pat. App. Pub. No. 2005/0244521 to Strickland et al.; and PCT Application Pub. No. WO 05/041699 to Quinter et al., each of which is incorporated herein by reference. In some instances, the flavoring agent may be provided in a spray-dried form or a liquid form.

The flavoring agent may be a volatile flavor component. As used herein, “volatile” refers to a chemical substance that forms a vapor readily at ambient temperatures (i.e., a chemical substance that has a high vapor pressure at a given temperature relative to a nonvolatile substance). Typically, a volatile flavor component has a molecular weight below about 400 Da, and often include at least one carbon-carbon double bond, carbon-oxygen double bond, or both. In one embodiment, the at least one volatile flavor component comprises one or more alcohols, aldehydes, aromatic hydrocarbons, ketones, esters, terpenes, terpenoids, or a combination thereof. Non-limiting examples of aldehydes include vanillin, ethyl vanillin, p-anisaldehyde, hexanal, furfural, isovaleraldehyde, cuminaldehyde, benzaldehyde, and citronellal. Non-limiting examples of ketones include 1-hydroxy-2-propanone and 2-hydroxy-3-methyl-2-cyclopentenone-1-one. Non-limiting examples of esters include allyl hexanoate, ethyl heptanoate, ethyl hexanoate, isoamyl acetate, and 3-methylbutyl acetate. Non-limiting examples of terpenes include sabinene, limonene, gamma-terpinene, beta-farnesene, nerolidol, thujone, myrcene, geraniol, nerol, citronellol, linalool, and eucalyptol. In one embodiment, the at least one volatile flavor component comprises one or more of ethyl vanillin, cinnamaldehyde, sabinene, limonene, gamma-terpinene, beta-farnesene, or citral. In one embodiment, the at least one volatile flavor component comprises ethyl vanillin.

Filler

The material or composition as described herein may include at least one particulate filler component. Such particulate fillers may fulfill multiple functions, such as enhancing certain organoleptic properties such as texture and mouthfeel, enhancing cohesiveness or compressibility of the product, and the like. Generally, the fillers are porous particulate materials and are cellulose-based. For example, suitable particulate fillers are any non-tobacco plant material or derivative thereof, including cellulose materials derived from such sources. Examples of cellulosic non-tobacco plant material include cereal grains (e.g., maize, oat, barley, rye, buckwheat, and the like), sugar beet (e.g., FIBREX® brand filler available from International Fiber Corporation), bran fiber, and mixtures thereof. Non-limiting examples of derivatives of non-tobacco plant material include starches (e.g., from potato, wheat, rice, corn), natural cellulose, and modified cellulosic materials. Additional examples of potential particulate fillers include maltodextrin, dextrose, calcium carbonate, calcium phosphate, lactose, mannitol, xylitol, and sorbitol. Combinations of fillers can also be used.

“Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the mixture based on the ability of the starch material to impart a specific organoleptic property to composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications, and are considered to be “genetically modified” starches. Other starches are obtained and subsequently modified by chemical, enzymatic, or physical means. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, acetylation, hydroxypropylation, and/or partial hydrolysis. Enzymatic treatment includes subjecting native starches to enzyme isolates or concentrates, microbial enzymes, and/or enzymes native to plant materials, e.g., amylase present in corn kernels to modify corn starch. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, starch sodium octenyl succinate.

In some embodiments, the particulate filler component is a cellulose material or cellulose derivative. One particularly suitable particulate filler component for use in the products described herein is microcrystalline cellulose (“MCC”). The MCC may be synthetic or semi-synthetic, or it may be obtained entirely from natural celluloses. The MCC may be selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof. In one embodiment, the mixture comprises MCC as the particulate filler component. The quantity of MCC present in the mixture as described herein may vary according to the desired properties.

The amount of particulate filler can vary, but is typically up to about 75 percent of the material by weight, based on the total weight of the material. A typical range of particulate filler (e.g., MCC) within the material can be from about 10 to about 75 percent by total weight of the mixture, for example, from about 10, about 15, about 20, about 25, or about 30, to about 35, about 40, about 45, or about 50 weight percent (e.g., about 20 to about 50 weight percent or about 25 to about 45 weight percent). In certain embodiments, the amount of particulate filler is at least about 10 percent by weight, such as at least about 20 percent, or at least about 25 percent, or at least about 30 percent, or at least about 35 percent, or at least about 40 percent, based on the total weight of the material.

In one embodiment, the particulate filler further comprises a cellulose derivative or a combination of such derivatives. In some embodiments, the mixture comprises from about 1 to about 10% of the cellulose derivative by weight, based on the total weight of the mixture, with certain embodiments comprising about 1 to about 5% by weight of cellulose derivative. In certain embodiments, the cellulose derivative is a cellulose ether (including carboxyalkyl ethers), meaning a cellulose polymer with the hydrogen of one or more hydroxyl groups in the cellulose structure replaced with an alkyl, hydroxyalkyl, or aryl group. Non-limiting examples of such cellulose derivatives include methylcellulose, hydroxypropylcellulose (“HPC”), hydroxypropylmethylcellulose (“HPMC”), hydroxyethyl cellulose, and carboxymethylcellulose (“CMC”). In one embodiment, the cellulose derivative is one or more of methylcellulose, HPC, HPMC, hydroxyethyl cellulose, and CMC. In one embodiment, the cellulose derivative is HPC. In some embodiments, the mixture comprises from about 1 to about 3% HPC by weight, based on the total weight of the material.

Tobacco Material

In some embodiments, the material or composition of the product may include a tobacco material. The tobacco material can vary in species, type, and form. Generally, the tobacco material is obtained from for a harvested plant of the Nicotiana species. Example Nicotiana species include N. tabacum, N. rustica, N. alata, N. arentsii, N. excelsior, N. forgetiana, N. glauca, N. glutinosa, N. gossei, N. kawakamii, N. knightiana, N. langsdorffi, N. otophora, N. setchelli, N. sylvestris, N. tomentosa, N. tomentosiformis, N. undulata, N. x sanderae, N. africana, N. amplexicaulis, N. benavidesii, N. bonariensis, N. debneyi, N. longiflora, N. maritina, N. megalosiphon, N. occidentalis, N. paniculata, N. plumbaginifolia, N. raimondii, N. rosulata, N. simulans, N. stocktonii, N. suaveolens, N. umbratica, N. velutina, N. wigandioides, N. acaulis, N. acuminata, N. attenuata, N. benthamiana, N. cavicola, N. clevelandii, N. cordifolia, N. corymbosa, N. fragrans, N. goodspeedii, N. linearis, N. miersii, N. nudicaulis, N. obtusifolia, N. occidentalis subsp. Hersperis, N. pauciflora, N. petunioides, N. quadrivalvis, N. repanda, N. rotundifolia, N. solanifolia, and N. spegazzinii. Various representative other types of plants from the Nicotiana species are set forth in Goodspeed, The Genus Nicotiana, (Chonica Botanica) (1954); U.S. Pat. No. 4,660,577 to Sensabaugh, Jr. et al.; 5,387,416 to White et al., U.S. Pat. No. 7,025,066 to Lawson et al.; U.S. Pat. No. 7,798,153 to Lawrence, Jr. and U.S. Pat. No. 8,186,360 to Marshall et al.; each of which is incorporated herein by reference. Descriptions of various types of tobaccos, growing practices and harvesting practices are set forth in Tobacco Production, Chemistry and Technology, Davis et al. (Eds.) (1999), which is incorporated herein by reference.

Nicotiana species from which suitable tobacco materials can be obtained can be derived using genetic-modification or crossbreeding techniques (e.g., tobacco plants can be genetically engineered or crossbred to increase or decrease production of components, characteristics or attributes). See, for example, the types of genetic modifications of plants set forth in U.S. Pat. No. 5,539,093 to Fitzmaurice et al.; U.S. Pat. No. 5,668,295 to Wahab et al.; U.S. Pat. No. 5,705,624 to Fitzmaurice et al.; U.S. Pat. No. 5,844,119 to Weigl; U.S. Pat. No. 6,730,832 to Dominguez et al.; U.S. Pat. No. 7,173,170 to Liu et al.; U.S. Pat. No. 7,208,659 to Colliver et al. and U.S. Pat. No. 7,230,160 to Benning et al.; US Patent Appl. Pub. No. 2006/0236434 to Conkling et al.; and PCT WO2008/103935 to Nielsen et al. See, also, the types of tobaccos that are set forth in U.S. Pat. No. 4,660,577 to Sensabaugh, Jr. et al.; 5,387,416 to White et al.; and U.S. Pat. No. 6,730,832 to Dominguez et al., each of which is incorporated herein by reference.

The Nicotiana species can, in some embodiments, be selected for the content of various compounds that are present therein. For example, plants can be selected on the basis that those plants produce relatively high quantities of one or more of the compounds desired to be isolated therefrom. In certain embodiments, plants of the Nicotiana species (e.g., Galpao commun tobacco) are specifically grown for their abundance of leaf surface compounds. Tobacco plants can be grown in greenhouses, growth chambers, or outdoors in fields, or grown hydroponically.

Various parts or portions of the plant of the Nicotiana species can be included within a mixture as disclosed herein. For example, virtually all of the plant (e.g., the whole plant) can be harvested, and employed as such. Alternatively, various parts or pieces of the plant can be harvested or separated for further use after harvest. For example, the flower, leaves, stem, stalk, roots, seeds, and various combinations thereof, can be isolated for further use or treatment. In some embodiments, the tobacco material comprises tobacco leaf (lamina). The material disclosed herein can include processed tobacco parts or pieces, cured and aged tobacco in essentially natural lamina and/or stem form, a tobacco extract, extracted tobacco pulp (e.g., using water as a solvent), or a mixture of the foregoing (e.g., a mixture that combines extracted tobacco pulp with granulated cured and aged natural tobacco lamina).

In certain embodiments, the tobacco material comprises solid tobacco material selected from the group consisting of lamina and stems. The tobacco that is used for the material most preferably includes tobacco lamina, or a tobacco lamina and stem mixture (of which at least a portion is smoke-treated). Portions of the tobaccos within the material may have processed forms, such as processed tobacco stems (e.g., cut-rolled stems, cut-rolled-expanded stems or cut-puffed stems), or volume expanded tobacco (e.g., puffed tobacco, such as dry ice expanded tobacco (DIET)). See, for example, the tobacco expansion processes set forth in U.S. Pat. No. 4,340,073 to de la Burde et al.; U.S. Pat. No. 5,259,403 to Guy et al.; and 5,908,032 to Poindexter, et al.; and U.S. Pat. No. 7,556,047 to Poindexter, et al., all of which are incorporated by reference. In addition, the material optionally may incorporate tobacco that has been fermented. See, also, the types of tobacco processing techniques set forth in PCT WO2005/063060 to Atchley et al., which is incorporated herein by reference.

The tobacco material is typically used in a form that can be described as particulate (i.e., shredded, ground, granulated, or powder form). The manner by which the tobacco material is provided in a finely divided or powder type of form may vary. Preferably, plant parts or pieces are comminuted, ground or pulverized into a particulate form using equipment and techniques for grinding, milling, or the like. Most preferably, the plant material is relatively dry in form during grinding or milling, using equipment such as hammer mills, cutter heads, air control mills, or the like. For example, tobacco parts or pieces may be ground or milled when the moisture content thereof is less than about 15 weight percent or less than about 5 weight percent. Most preferably, the tobacco material is employed in the form of parts or pieces that have an average particle size between 1.4 millimeters and 250 microns. In some instances, the tobacco particles may be sized to pass through a screen mesh to obtain the particle size range required. If desired, air classification equipment may be used to ensure that small sized tobacco particles of the desired sizes, or range of sizes, may be collected. If desired, differently sized pieces of granulated tobacco may be mixed together.

The manner by which the tobacco is provided in a finely divided or powder type of form may vary. Preferably, tobacco parts or pieces are comminuted, ground or pulverized into a powder type of form using equipment and techniques for grinding, milling, or the like. Most preferably, the tobacco is relatively dry in form during grinding or milling, using equipment such as hammer mills, cutter heads, air control mills, or the like. For example, tobacco parts or pieces may be ground or milled when the moisture content thereof is less than about 15 weight percent to less than about 5 weight percent. For example, the tobacco plant or portion thereof can be separated into individual parts or pieces (e.g., the leaves can be removed from the stems, and/or the stems and leaves can be removed from the stalk). The harvested plant or individual parts or pieces can be further subdivided into parts or pieces (e.g., the leaves can be shredded, cut, comminuted, pulverized, milled or ground into pieces or parts that can be characterized as filler-type pieces, granules, particulates or fine powders). The plant, or parts thereof, can be subjected to external forces or pressure (e.g., by being pressed or subjected to roll treatment). When carrying out such processing conditions, the plant or portion thereof can have a moisture content that approximates its natural moisture content (e.g., its moisture content immediately upon harvest), a moisture content achieved by adding moisture to the plant or portion thereof, or a moisture content that results from the drying of the plant or portion thereof. For example, powdered, pulverized, ground or milled pieces of plants or portions thereof can have moisture contents of less than about 25 weight percent, often less than about 20 weight percent, and frequently less than about 15 weight percent.

For the preparation of oral products, it is typical for a harvested plant of the Nicotiana species to be subjected to a curing process. The tobacco materials incorporated within the material for inclusion within products as disclosed herein are those that have been appropriately cured and/or aged. Descriptions of various types of curing processes for various types of tobaccos are set forth in Tobacco Production, Chemistry and Technology, Davis et al. (Eds.) (1999). Examples of techniques and conditions for curing flue-cured tobacco are set forth in Nestor et al., Beitrage Tabakforsch. Int, 20, 467-475 (2003) and U.S. Pat. No. 6,895,974 to Peele, which are incorporated herein by reference. Representative techniques and conditions for air curing tobacco are set forth in U.S. Pat. No. 7,650,892 to Groves et al.; Roton et al., Beitrage Tabakforsch. Int, 21, 305-320 (2005) and Staaf et al., Beitrage Tabakforsch. Int, 21, 321-330 (2005), which are incorporated herein by reference. Certain types of tobaccos can be subjected to alternative types of curing processes, such as fire curing or sun curing.

In certain embodiments, tobacco materials that can be employed include flue-cured or Virginia (e.g., K326), burley, sun-cured (e.g., Indian Kurnool and Oriental tobaccos, including Katerini, Prelip, Komotini, Xanthi and Yambol tobaccos), Maryland, dark, dark-fired, dark air cured (e.g., Madole, Passanda, Cubano, Jatin and Bezuki tobaccos), light air cured (e.g., North Wisconsin and Galpao tobaccos), Indian air cured, Red Russian and Rustica tobaccos, as well as various other rare or specialty tobaccos and various blends of any of the foregoing tobaccos.

The tobacco material may also have a so-called “blended” form. For example, the tobacco material may include a mixture of parts or pieces of flue-cured, burley (e.g., Malawi burley tobacco) and Oriental tobaccos (e.g., as tobacco composed of, or derived from, tobacco lamina, or a mixture of tobacco lamina and tobacco stem). For example, a representative blend may incorporate about 30 to about 70 parts burley tobacco (e.g., lamina, or lamina and stem), and about 30 to about 70 parts flue cured tobacco (e.g., stem, lamina, or lamina and stem) on a dry weight basis. Other example tobacco blends incorporate about 75 parts flue-cured tobacco, about 15 parts burley tobacco, and about 10 parts Oriental tobacco; or about 65 parts flue-cured tobacco, about 25 parts burley tobacco, and about 10 parts Oriental tobacco; or about 65 parts flue-cured tobacco, about 10 parts burley tobacco, and about 25 parts Oriental tobacco; on a dry weight basis. Other example tobacco blends incorporate about 20 to about 30 parts Oriental tobacco and about 70 to about 80 parts flue-cured tobacco on a dry weight basis.

Tobacco materials used in the present disclosure can be subjected to, for example, fermentation, bleaching, and the like. If desired, the tobacco materials can be, for example, irradiated, pasteurized, or otherwise subjected to controlled heat treatment. Such treatment processes are detailed, for example, in U.S. Pat. No. 8,061,362 to Mua et al., which is incorporated herein by reference. In certain embodiments, tobacco materials can be treated with water and an additive capable of inhibiting reaction of asparagine to form acrylamide upon heating of the tobacco material (e.g., an additive selected from the group consisting of lysine, glycine, histidine, alanine, methionine, cysteine, glutamic acid, aspartic acid, proline, phenylalanine, valine, arginine, compositions incorporating di- and trivalent cations, asparaginase, certain non-reducing saccharides, certain reducing agents, phenolic compounds, certain compounds having at least one free thiol group or functionality, oxidizing agents, oxidation catalysts, natural plant extracts (e.g., rosemary extract), and combinations thereof. See, for example, the types of treatment processes described in U.S. Pat. Nos. 8,434,496, 8,944,072, and 8,991,403 to Chen et al., which are all incorporated herein by reference. In certain embodiments, this type of treatment is useful where the original tobacco material is subjected to heat in the processes previously described.

In some embodiments, the type of tobacco material is selected such that it is initially visually lighter in color than other tobacco materials to some degree (e.g., whitened or bleached). Tobacco pulp can be whitened in certain embodiments according to any means known in the art. For example, bleached tobacco material produced by various whitening methods using various bleaching or oxidizing agents and oxidation catalysts can be used. Example oxidizing agents include peroxides (e.g., hydrogen peroxide), chlorite salts, chlorate salts, perchlorate salts, hypochlorite salts, ozone, ammonia, potassium permanganate, and combinations thereof. Example oxidation catalysts are titanium dioxide, manganese dioxide, and combinations thereof. Processes for treating tobacco with bleaching agents are discussed, for example, in U.S. Pat. No. 787,611 to Daniels, Jr.; U.S. Pat. No. 1,086,306 to Oelenheinz; U.S. Pat. No. 1,437,095 to Delling; U.S. Pat. No. 1,757,477 to Rosenhoch; U.S. Pat. No. 2,122,421 to Hawkinson; U.S. Pat. No. 2,148,147 to Baier; U.S. Pat. No. 2,170,107 to Baier; U.S. Pat. No. 2,274,649 to Baier; U.S. Pat. No. 2,770,239 to Prats et al.; U.S. Pat. No. 3,612,065 to Rosen; U.S. Pat. No. 3,851,653 to Rosen; U.S. Pat. No. 3,889,689 to Rosen; U.S. Pat. No. 3,943,940 to Minami; U.S. Pat. No. 3,943,945 to Rosen; U.S. Pat. No. 4,143,666 to Rainer; U.S. Pat. No. 4,194,514 to Campbell; U.S. Pat. Nos. 4,366,823, 4,366,824, and 4,388,933 to Rainer et al.; U.S. Pat. No. 4,641,667 to Schmekel et al.; U.S. Pat. No. 5,713,376 to Berger; U.S. Pat. No. 9,339,058 to Byrd Jr. et al.; 9,420,825 to Beeson et al.; and U.S. Pat. No. 9,950,858 to Byrd Jr. et al.; as well as in US Pat. App. Pub. Nos. 2012/0067361 to Bjorkholm et al.; 2016/0073686 to Crooks; 2017/0020183 to Bjorkholm; and 2017/0112183 to Bjorkholm, and in PCT Publ. Appl. Nos. WO1996/031255 to Giolvas and WO2018/083114 to Bjorkholm, all of which are incorporated herein by reference.

In some embodiments, the whitened tobacco material can have an ISO brightness of at least about 50%, at least about 60%, at least about 65%, at least about 70%, at least about 75%, or at least about 80%. In some embodiments, the whitened tobacco material can have an ISO brightness in the range of about 50% to about 90%, about 55% to about 75%, or about 60% to about 70%. ISO brightness can be measured according to ISO 3688:1999 or ISO 2470-1:2016.

In some embodiments, the whitened tobacco material can be characterized as lightened in color (e.g., “whitened”) in comparison to an untreated tobacco material. White colors are often defined with reference to the International Commission on Illumination's (CIE's) chromaticity diagram. The whitened tobacco material can, in certain embodiments, be characterized as closer on the chromaticity diagram to pure white than an untreated tobacco material.

In various embodiments, the tobacco material can be treated to extract a soluble component of the tobacco material therefrom. “Tobacco extract” as used herein refers to the isolated components of a tobacco material that are extracted from solid tobacco pulp by a solvent that is brought into contact with the tobacco material in an extraction process. Various extraction techniques of tobacco materials can be used to provide a tobacco extract and tobacco solid material. See, for example, the extraction processes described in US Pat. Appl. Pub. No. 2011/0247640 to Beeson et al., which is incorporated herein by reference. Other example techniques for extracting components of tobacco are described in U.S. Pat. No. 4,144,895 to Fiore; U.S. Pat. No. 4,150,677 to Osborne, Jr. et al.; 4,267,847 to Reid; U.S. Pat. No. 4,289,147 to Wildman et al.; U.S. Pat. No. 4,351,346 to Brummer et al.; U.S. Pat. No. 4,359,059 to Brummer et al.; U.S. Pat. No. 4,506,682 to Muller; U.S. Pat. No. 4,589,428 to Keritsis; U.S. Pat. No. 4,605,016 to Soga et al.; U.S. Pat. No. 4,716,911 to Poulose et al.; U.S. Pat. No. 4,727,889 to Niven, Jr. et al.; 4,887,618 to Bernasek et al.; U.S. Pat. No. 4,941,484 to Clapp et al.; U.S. Pat. No. 4,967,771 to Fagg et al.; U.S. Pat. No. 4,986,286 to Roberts et al.; U.S. Pat. No. 5,005,593 to Fagg et al.; U.S. Pat. No. 5,018,540 to Grubbs et al.; U.S. Pat. No. 5,060,669 to White et al.; U.S. Pat. No. 5,065,775 to Fagg; U.S. Pat. No. 5,074,319 to White et al.; U.S. Pat. No. 5,099,862 to White et al.; U.S. Pat. No. 5,121,757 to White et al.; U.S. Pat. No. 5,131,414 to Fagg; U.S. Pat. No. 5,131,415 to Munoz et al.; U.S. Pat. No. 5,148,819 to Fagg; U.S. Pat. No. 5,197,494 to Kramer; U.S. Pat. No. 5,230,354 to Smith et al.; U.S. Pat. No. 5,234,008 to Fagg; U.S. Pat. No. 5,243,999 to Smith; U.S. Pat. No. 5,301,694 to Raymond et al.; U.S. Pat. No. 5,318,050 to Gonzalez-Parra et al.; U.S. Pat. No. 5,343,879 to Teague; U.S. Pat. No. 5,360,022 to Newton; U.S. Pat. No. 5,435,325 to Clapp et al.; U.S. Pat. No. 5,445,169 to Brinkley et al.; U.S. Pat. No. 6,131,584 to Lauterbach; U.S. Pat. No. 6,298,859 to Kierulff et al.; U.S. Pat. No. 6,772,767 to Mua et al.; and U.S. Pat. No. 7,337,782 to Thompson, all of which are incorporated by reference herein.

Typical inclusion ranges for tobacco materials can vary depending on the nature and type of the tobacco material, and the intended effect on the final mixture, with an example range of up to about 30% by weight (or up to about 20% by weight or up to about 10% by weight or up to about 5% by weight), based on total weight of the mixture (e.g., about 0.1 to about 15% by weight).

It should be noted that inclusion of a tobacco material into the compositions and products described herein is meant to be optional and is not required. In some embodiments, oral products as described herein can generally be characterized as being tobacco free-alternatives. For example, in some embodiments, oral products of the present disclosure may be said to be completely free or substantially free of tobacco material (other than purified nicotine as an active ingredient). Oral products that are referred to as “completely free of” or “substantially free of” a tobacco material herein are meant to refer to oral products that can be characterized as having less than about 1.0% by weight, less than about 0.5% by weight, less than about 0.1% by weight of tobacco material, or 0% by weight of tobacco material.

Salts

In some embodiments, the reconstituted tobacco may further comprise a salt (e.g., alkali metal salts), typically employed in an amount sufficient to provide desired sensory attributes to the reconstituted tobacco. Non-limiting examples of suitable salts include sodium chloride, potassium chloride, ammonium chloride, flour salt, and the like. When present, a representative amount of salt is about 0.5 percent by weight or more, about 1.0 percent by weight or more, or at about 1.5 percent by weight or more, but will typically make up about 10 percent or less of the total weight of the reconstituted tobacco, or about 7.5 percent or less or about 5 percent or less (e.g., about 0.5 to about 5 percent by weight).

Sweeteners

The material or composition of the product can further include one or more sweeteners. The sweeteners can be any sweetener or combination of sweeteners, in natural or artificial form, or as a combination of natural and artificial sweeteners. Examples of natural sweeteners include isomaltulose, fructose, sucrose, glucose, maltose, mannose, galactose, lactose, stevia, honey, and the like. Examples of artificial sweeteners include sucralose, maltodextrin, saccharin, aspartame, acesulfame K, neotame and the like. In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). When present, a representative amount of sweetener may make up from about 0.1 to about 20 percent or more of the of the mixture by weight, for example, from about 0.1 to about 1%, from about 1 to about 5%, from about 5 to about 10%, or from about 10 to about 20% of the mixture on a weight basis, based on the total weight of the reconstituted tobacco.

Additional Binding Agents

In one or more embodiments, the material or composition may include one or more additional binding agents, e.g., in additional to the one or more binding agents detailed herein above. A binder (or combination of binders) may be employed in certain embodiments, in amounts sufficient to provide the desired physical attributes and physical integrity to the mixture. Binders also often function as thickening or gelling agents. Typical binders can be organic or inorganic, or a combination thereof. Representative binders include modified cellulose, povidone, sodium alginate, starch-based binders, pectin, carrageenan, pullulan, zein, and the like, and combinations thereof. In some embodiments, the binder comprises pectin or carrageenan or combinations thereof.

An additional binder may be employed in amounts sufficient to provide the desired physical attributes and physical integrity to the reconstituted tobacco. The amount of binder utilized in the reconstituted tobacco can vary, but is typically up to about 30 weight percent, and certain embodiments are characterized by a binder content of at least about 0.1% by weight, such as about 1 to about 30% by weight, or about 5 to about 10% by weight, based on the total weight of the reconstituted tobacco.

In certain embodiments, the additional binder includes a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, and combinations thereof. When present, natural gum binder materials are typically present in an amount of up to about 5% by weight, for example, from about 0.1, about 0.2, about 0.3, about 0.4, about 0.5, about 0.6, about 0.7, about 0.8, about 0.9, or about 1%, to about 2, about 3, about 4, or about 5% by weight, based on the total weight of the reconstituted tobacco.

Humectants

In certain embodiments, one or more humectants may be employed in the products of the present disclosure. Examples of humectants include, but are not limited to, polyols such as glycerin, propylene glycol, and the like. Where included, the humectant is typically provided in an amount sufficient to provide desired moisture attributes to the mixture. Further, in some instances, the humectant may impart desirable flow characteristics to the mixture for depositing in a mold. When present, a humectant will typically make up about 5% or less of the weight of the reconstituted tobacco (e.g., from about 0.5 to about 5% by weight). When present, a representative amount of humectant is about 0.1% to about 1% by weight, or about 1% to about 5% by weight, based on the total weight of the reconstituted tobacco.

Buffering Agents

In certain embodiments, the mixture or composition of the products of the present disclosure can comprise pH adjusters or buffering agents. Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Where present, the buffering agent is typically present in an amount less than about 5 percent based on the weight of the reconstituted tobacco, for example, from about 0.5% to about 5%, such as, e.g., from about 0.75% to about 4%, from about 0.75% to about 3%, or from about 1% to about 2% by weight, based on the total weight of the reconstituted tobacco. Non-limiting examples of suitable buffers include alkali metals acetates, glycinates, phosphates, glycerophosphates, citrates, carbonates, hydrogen carbonates, borates, or mixtures thereof.

Colorants

A colorant may be employed in amounts sufficient to provide the desired physical attributes to the reconstituted tobacco. Examples of colorants include various dyes and pigments, such as caramel coloring and titanium dioxide. The amount of colorant utilized in the mixture can vary, but when present is typically up to about 3 weight percent, such as from about 0.1%, about 0.5%, or about 1%, to about 3% by weight, based on the total weight of the reconstituted tobacco.

Moisture Content

In some embodiments, the material may include a content of water. The water content of the composition within the product, prior to use by a consumer of the product, may vary according to the desired properties. Typically, the composition, as present within the product prior to insertion into the mouth of the user, can comprise less than 60%, less than 50%, less than 40%, or at least about 10%, at least about 20%, or at least about 30% by weight of water, based on the total weight of the product. For example, total water content in the composition and/or product may be in the range of about 10% to about 60%, about 20% to about 60%, about 30% to about 60%, or about 40% to about 60% by weight of water. Without intending to be bound by theory it is believed that compositions and products having a high water content (e.g., in the range of about 30% to about 60%), in particular, can benefit from the increased water stability of the agglomerated particles. In some embodiments, the compositions and products may include at least 10%, at least 20%, at least 30%, at least 40%, or at least 50% by weight water (e.g., about 40% to about 80% or about 50% to about 75% or about 50% to about 65%).

Organic Acid

In some embodiments, the material may include a content of one or more organic acids. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific ingredient as opposed to merely being inherently present as a component of another ingredient (e.g., the small amount of organic acid which may inherently be present in an ingredient such as a tobacco material). In some embodiments, the one or more organic acids are added neat (i.e., in their free acid, native solid or liquid form) or as a solution in, e.g., water. In some embodiments, the one or more organic acids are added in the form of a salt, as described herein below.

In some embodiments, the organic acid is an alkyl carboxylic acid. Non-limiting examples of alkyl carboxylic acids include formic acid, acetic acid, propionic acid, octanoic acid, nonanoic acid, decanoic acid, undecanoic acid, dodecanoic acid, stearic acid, oleic acid, linoleic acid, linolenic acid, and the like. In some embodiments, the organic acid is an alkyl sulfonic acid. Non-limiting examples of alkyl sulfonic acids include propanesulfonic acid and octanesulfonic acid. In some embodiments, the alkyl carboxylic or sulfonic acid is substituted with one or more hydroxyl groups. Non-limiting examples include glycolic acid, 4-hydroxybutyric acid, and lactic acid. In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like. In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid. In some embodiments, the organic acid is citric acid, malic acid, tartaric acid, octanoic acid, benzoic acid, a toluic acid, salicylic acid, or a combination thereof. In some embodiments, the organic acid is benzoic acid. In some embodiments, the organic acid is citric acid. In alternative embodiments, a portion, or even all, of the organic acid may be added in the form of a salt with an alkaline component, which may include, but is not limited to, nicotine. Non-limiting examples of suitable salts, e.g., for nicotine, include formate, acetate, propionate, isobutyrate, butyrate, alpha-methylbutyate, isovalerate, beta-methylvalerate, caproate, 2-furoate, phenylacetate, heptanoate, octanoate, nonanoate, oxalate, malonate, glycolate, benzoate, tartrate, levulinate, ascorbate, fumarate, citrate, malate, lactate, aspartate, salicylate, tosylate, succinate, pyruvate, and the like.

The amount of organic acid present in the compositions may vary. Generally, the compositions can comprise from 0 to about 10% by weight of organic acid, present as one or more organic acids, based on the total weight of the material.

Examples of even further types of additives that may be used in the present materials include thickening or gelling agents (e.g., fish gelatin), emulsifiers, oral care additives (e.g., thyme oil, eucalyptus oil, and zinc), preservatives (e.g., potassium sorbate and the like), disintegration aids, zinc or magnesium salts selected to be relatively water soluble for compositions with greater water solubility (e.g., magnesium or zinc gluconate) or selected to be relatively water insoluble for compositions with reduced water solubility (e.g., magnesium or zinc oxide), or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., US Pat. App. Pub. No. 2010/0291245 to Gao et al., and US Pat. App. Pub. No. 2007/0062549 to Holton, Jr. et al., each of which is incorporated herein by reference. Typical inclusion ranges for such additional additives can vary depending on the nature and function of the additive and the intended effect on the final mixture, with an example range of up to about 10% by weight, based on total weight of the substrate material (e.g., about 0.1 to about 5% by weight).

The aforementioned additives can be employed together (e.g., as additive formulations) or separately (e.g., individual additive components can be added at different stages involved in the preparation of the final substrate material). Furthermore, the aforementioned types of additives may be encapsulated as provided in the final product or material to be included within the final product. Example encapsulated additives are described, for example, in WO2010/132444 to Atchley, which has been previously incorporated by reference herein.

### Experimental

Aspects of the present invention are more fully illustrated by the following examples, which are set forth to illustrate certain aspects of the present invention and are not to be construed as limiting thereof.

Tobacco stem dust was treated with various binding agents to form an agglomerated tobacco material. The combined agglomerated tobacco materials were then treated (e.g., using a fixing method) to decrease the water solubility of the agglomerated particle fines, if necessary. Multiple samples were prepared using a combination of binding agents and fixing methods as described in Table 1, and as will be discussed in more detail herein.

TABLE 1

| Sample | | Stem/ | |
|---|---|---|---|
| No. | Binder | binder | Fixing method |
| 1 | 1.93% Alginate (Manucol LD) | 0.974 | CaCl₂ solution |
| 2 | 1.93% Alginate (Protanal 6650) | 1.008 | CaCl₂ solution |
| 3 | 1.93% Alginate (Protanal 6650) | 0.812 | CaCl₂ solution |
| 4 | 1.97% Manucol + 2.35% HPMC | 0.840 | 80° C. 2.05% |
| | | | CaCl₂ |
| 5 | 1.48% Chitosan in 1% acetic acid | 0.980 | NaHCO₃ |
| 6 | 5.24% solids PVA adhesive | 0.901 | Drying |
| 7 | 5.18% solids PVA adhesive | 0.875 | Drying |
| 8 | 10.35% solids PVA adhesive | 0.851 | Drying |
| 9 | 2.00% Et cellulose in ethanol | 0.807 | Drying |
| 10 | Palm kernel fat w/heat | 3.25 | Cooling |
| 11 | Palm kernel fat w/heat | 2.46 | Cooling |

Sample 1 is a 1.93% sodium alginate solution prepared by adding 9.88 g of Manucol LD to 503.3 g of water. The solution of Sample 1 was added to a content of tobacco stem dust in a weight ratio of tobacco stem dust to binding agent of 0.974 and allowed to hydrate for at least 30 minutes. After hydration, the combined tobacco stem dust and binding agent were observed to be in the form of a “dough” and were pressed through an 850-micron sieve. The sieved particles were then transferred to a beaker containing 104 g of a 4.8% calcium chloride solution (CaCl₂), stirred for several minutes, and allowed to agglomerate. The agglomerated tobacco particles were then poured onto a 106-micron sieve to drain and subsequently rinsed with deionized water. The wet agglomerated particles were then allowed to air dry for approximately 2 hours and then passed through an 850-micron sieve. The remaining agglomerated particles were then allowed to air dry overnight and were subsequently filtered through a stack of sieves to determine the particle size distribution of the agglomerated particles. Results showed that the size distribution of the agglomerated particles included 5.7% of particles having a particle size of less than 300 microns, 5.0% of particles having a particle size of between 300 and 425 microns, 17.0% of particles having a particle size of between 425 and 700 microns, and 72.1% having a particle size of greater than 700 microns.

A more severe test was also conducted to evaluate the sturdiness of the agglomerated particles. The agglomerated particles were poured over a stack of sieves ranging from 700 microns to 106 microns and sprayed with water until the particles were broken up and passed through the 700 micron sieve. In total, 160 g of water was sprayed on the 0.363 g of agglomerated particle fines. Most of the agglomerated particles passed through the 425-micron sieve but remained on the 300-micron sieve, evidencing that the agglomerated particles had a particle size greater than 300 microns. Next, the agglomerated particles were sprayed with 110 g of water on the 300-micron sieve and roughly half of the particles rinsed through the 300-micron sieve. Although the particles were eventually able to be broken up via application of water, this test indicated that the particles were robust enough to survive blending and pouching in a relatively dry state before adding water to the pouched product.

Sample 2 is a 1.93% sodium alginate solution prepared by adding 7.98 g of Protanal RF to 405.0 g of water and allowing the solution to hydrate. The solution of Sample 2 was added to a content of tobacco stem dust in a weight ratio of tobacco stem dust to binding agent of 1.008 and allowed to hydrate for at least 30 minutes. After hydration, the combined tobacco stem dust and binding agent were pressed through a 1.16 mm sieve and then an 850-micron sieve. The sieved particles were then transferred to a beaker containing 103 g of a 3% calcium chloride solution (CaCl₂) and allowed to cross-link the alginate for 5-10 minutes. The agglomerated particles were then poured onto a 425-micron sieve to drain and subsequently rinsed with deionized water. Most of the agglomerated particles remained on top of the 425-micron sieve after being rinsed with deionized water.

Sample 3 is a 1.93% sodium alginate solution prepared by adding 7.98 g of Protanal RF to 405.0 g of water and allowing the solution to hydrate. The solution of Sample 3 was added to a content of tobacco stem dust in a weight ratio of tobacco stem dust to binding agent of 0.812 and allowed to hydrate for at least 30 minutes. After hydration, the combined tobacco stem dust and binding agent were pressed through a 1.18 mm sieve. The sieved particles were then misted with a 4.9% calcium chloride solution (CaCl₂) and allowed to cross-link the alginate. After about 1 hour, the agglomerated particles were re-misted with the calcium chloride solution and left to dry overnight. The agglomerated particles were then re-misted with the calcium chloride solution and allowed to dry for 2 days. The remaining agglomerated particle fines were then filtered through a stack of sieves to determine the particle size distribution of the agglomerated particles. Results showed that the size distribution of the agglomerated particles included 34% of particles having a particle size of less than 300 microns, 14% of particles having a particle size of between 300 and 425 microns, and 52% of particles having a particle size of greater than 425 microns.

Sample 4 is a 1.93% Manucol/2.35% hydroxypropyl methyl cellulose (“HPMC”) solution prepared by adding 12.48 g of HPMC to 512 g of boiling water in a homogenizer while stirring. Next, the HPMC solution was stored in refrigerator for a week and then 2.0014 g of a Manucol LD solution was added to 99.70 g of the HPMC solution and stirred until all the sodium alginate solution was mixed into solution (approximately 1.5 hours). The solution of Sample 1 was added to a content of tobacco stem dust in a weight ratio of tobacco stem dust to binding agent of 0.840 and allowed to hydrate for at least 30 minutes. After hydration, the combined tobacco stem dust and binding agent were pressed through a 1.18 mm sieve on top of an 850-micron sieve. Most of the combined particles remained on the 850-micron sieve. The remaining particles were added to a 425-micron sieve on top of a 300-micron sieve, and very few particles passed through the 300-micron sieve. Then, the sieved particles were added to a beaker positioned on a hot plate containing 102 g of a 2.0% calcium chloride solution (CaCl₂) at a temperature of 80° C. and allowed to gel and cross-link the binder. The agglomerated particles were then poured onto a 250-micron sieve to drain and subsequently rinsed twice with deionized water. The remaining agglomerated particles were then allowed to air dry overnight and were subsequently filtered through a stack of sieves to determine the particle size distribution of the agglomerated particles. Results showed that the size distribution of the agglomerated particles included 62.4% of particles having a particle size of greater than 300 microns.

Sample 5 was prepared by adding 19.56 g of distilled white vinegar (5% acidity) to 99.90 g of water to form a 1% acetic acid solution. Next, 1.502 g of Chitosan was added to the acetic acid solution and stirred until completely dissolved (approximately 2-2.5 hours). The combined solution was stored in a refrigerator until used. The solution of Sample 5 was added to a content of tobacco stem dust in a weight ratio of tobacco stem dust to binding agent of 0.980 and allowed to hydrate for at least 30 minutes. After hydration, the combined tobacco stem dust and binding agent were pressed through a 1.18 mm sieve, and subsequently an 850-micron sieve. The remaining particles were combined and 1.01 g of dry calcium bicarbonate (NaHCO₃) was stirred into 4.59 g of the tobacco particle fines along with 0.92 g of deionized water to neutralize the acetic acid. The agglomerated particles were then pressed through the 1.18 mm sieve and the 850-micron sieve and allowed to dry overnight The remaining agglomerated particles were then allowed to air dry overnight and were subsequently filtered through a stack of sieves to determine the particle size distribution of the agglomerated particle fines. Results showed that the size distribution of the agglomerated particles included 58% of particles having a particle size of smaller than 300 microns, 17% of particles having a particle size between about 300 microns and 850 microns, and 25% of particles having a particle size of greater than 850 microns.

Sample 6 was prepared by diluting roughly 1 part of a polyvinyl acetate (PVA) emulsion with 9 parts water to provide a 5.24% solids PVA emulsion. The solution of Sample 6 was added to a content of tobacco stem dust in a weight ratio of tobacco stem dust to binding agent of 0.901 and allowed to hydrate for at least 30 minutes. After hydration, the combined tobacco stem dust and binding agent were pressed through a 1.18 mm sieve on top of an 850-micron sieve. Most of the agglomerated particles remained on the 850-micron sieve. The remaining particles were allowed to air dry briefly and then were passed through a 425-micron sieve, and subsequently a 300-micron sieve. Results showed that the size distribution of the agglomerated particles included 89% of particles having a particle size of greater than 300 microns.

Sample 7 was prepared by diluting roughly 1 part of a polyvinyl acetate (PVA) emulsion with 9 parts water to provide a 5.18% solids PVA emulsion. The solution of Sample 7 was added to a content of tobacco stem dust in a weight ratio of tobacco stem dust to binding agent of 0.875 and allowed to hydrate for at least 30 minutes. After hydration, the combined tobacco stem dust and binding agent were pressed through a 1.18 mm sieve on top of an 850-micron sieve. Most of the agglomerated particles remained on the 850-micron sieve. The remaining particles were allowed to air dry briefly and then were passed through a 425-micron sieve, and subsequently a 300-micron sieve. Results showed that the size distribution of the agglomerated particles included 37% of particles having a particle size of greater than 850 microns, 37% of particles having a particle size of greater than 300 microns, and 26% of particles having a particle size of less than 300 microns.

Sample 8 was prepared by diluting 1.19 g of a polyvinyl acetate (PVA) emulsion with water to a final weight of 5.98 g to provide a roughly 10% solids PVA emulsion. The solution of Sample 8 was added to a content of tobacco stem dust in a weight ratio of tobacco stem dust to binding agent of 0.851 and allowed to hydrate for at least 30 minutes. After hydration, the combined tobacco stem dust and binding agent were pressed through a 1.18 mm sieve on top of an 850-micron sieve. Most of the agglomerated particles remained on the 850-micron sieve. The remaining particles were allowed to air dry briefly and then were passed through a 425-micron sieve, and subsequently a 300-micron sieve. Results showed that the size distribution of the agglomerated particles included 8% of particles having a particle size of greater than 850 microns, 82% of particles having a particle size of greater than 300 microns, and 10% of particles having a particle size of less than 300 microns.

Sample 9 was prepared by diluting 1.0094 g of an ethyl cellulose to 50.42 total grams by adding 200 proof USP ethanol while stirring. The 2.00% ethyl cellulose solution was refrigerated until use. The solution of Sample 9 was added to a content of tobacco stem dust in a weight ratio of tobacco stem dust to binding agent of 0.807. Next, the combined tobacco stem dust and binding agent were pressed through a 1.18 mm sieve on top of an 850-micron sieve. Most of the agglomerated particles remained on the 850-micron sieve. The remaining particles were allowed to air dry briefly and then were passed through a 425-micron sieve, and subsequently a 300-micron sieve. Results showed that the size distribution of the agglomerated particles included 34% of particles having a particle size of greater than 850 microns, 55% of particles having a particle size of greater than 300 microns, and 11% of particles having a particle size of less than 300 microns.

Sample 10 was prepared by mixing 172 g of tobacco particle fines with 53 g of partially hydrogenated palm kernel fat in a mixer. The mixture included a content of tobacco stem dust in a weight ratio of tobacco stem dust to binding agent of 3.25. The mixture was whisked at room temperature; however, sufficient mixing of the tobacco stem dust and palm kernel fat was not observed. Next, the mixing bowl including the mixture was heated to a temperature in the range of about 91° F. to about 93° F. and more complete mixing was observed. The sample was then cooled and pressed through a 300-micron sieve. Approximately 17.9% of the agglomerated particles remained on the 300-micron sieve.

Sample 11 was prepared by adding 70 g of palm kernel fat to the final combined mixture provided in sample 10. The mixture included a content of tobacco stem dust in a weight ratio of tobacco stem dust to binding agent of 2.46. The mixture was heated to a temperature in the range of about 82° F. to about 83° F. and whisked for several minutes. Sample 11 was then cooled and added to a 425-micron sieve on top of a 300-micron sieve. Results showed that the size distribution of the agglomerated particles included 7% of particles having a particle size of greater than 425 microns, 31% of particles having a particle size of greater than 300 microns, and 62% of particles having a particle size of less than 300 microns. While the majority of the agglomerated particles in Samples 10 and 11 were observed to have an average particle size of less than 300 microns, it should be noted that addition of the palm kernel fat led to significantly reduced dusting. Other plant-based oil materials may provide improved agglomeration performance.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A method of preparing an agglomerated botanical material having an increased water stability, comprising:
combining botanical particle fines having an average particle size of about 300 microns or less with a binding agent, the binding agent being selected from the group consisting of alginates, chitosan, water-insoluble polymers, plant oils having a melting point above room temperature, and combinations thereof;
forming agglomerated botanical particles from the combined botanical particle fines and binding agent, the agglomerated botanical particles having an average particle size greater than the tobacco particle fines; and
optionally, treating the agglomerated botanical particles to increase the water stability thereof.

2. The method of claim 1, wherein the combining step comprises combining the botanical particle fines with the binding agent at a weight ratio of botanical particle fines to binding agent of from about 0.5:1 to about 5:1.

3. The method of claim 1, wherein the combining step comprises combining the botanical particle fines with the binding agent at a weight ratio of botanical particle fines to binding agent of from about 0.8:1 to about 3.25:1.

4. The method of claim 1, wherein the combining step comprises combining the botanical particle fines with the binding agent at a weight ratio of botanical particle fines to binding agent of about 3.25 or less.

5. The method of claim 1, wherein the agglomerated botanical particles have an average particle size greater than about 350 microns.

6. The method of claim 1, wherein the agglomerated botanical particles have an average particle size greater than about 400 microns.

7. The method of claim 1, wherein the agglomerated botanical particles have an average particle size greater than about 500 microns.

8. The method of claim 1, wherein the binding agent is a water-insoluble polymer selected from group consisting of polyvinyl ester polymers and polymeric cellulose derivatives.

9. The method of claim 1, wherein the binding agent is a water-insoluble polymer selected from the group consisting of polyvinyl acetate and ethyl cellulose.

10. The method of claim 1, wherein the binding agent is a water-insoluble polymer in the form of an aqueous emulsion, and the method further comprises drying the agglomerated botanical particles.

11. The method of claim 1, wherein the binding agent is a plant oil having a melting point above room temperature, and wherein the combining is optionally conducted at elevated temperature.

12. The method of claim 11, further comprises cooling the agglomerated botanical particles to solidify the plant oil.

13. The method of claim 1, wherein the binding agent is chitosan or an alginate salt, optionally combined with hydroxypropyl methyl cellulose.

14. The method of claim 1, wherein the binding agent is chitosan and the optional treating step comprises treating the agglomerated botanical particles to increase the pH to about 7 or higher.

15. The method of claim 1, wherein the binding agent is an alginate salt and the optional treating step comprises treating the agglomerated botanical particles with a divalent cation.

16. The method of claim 15, wherein the divalent cation comprises calcium ions.

17. The method of claim 1, wherein the binding agent is an alginate salt combined with hydroxypropyl methyl cellulose and the optional treating step comprises treating the agglomerated botanical particles with a divalent cation solution at elevated temperature.

18. The method of claim 1, wherein the botanical particle fines comprise stems of a plant of the Nicotiana species.

19. A product adapted for oral use comprising a plurality of agglomerated botanical particles, each agglomerated botanical particle comprising a plurality of botanical particle fines having an average particle size of about 300 microns and a binding agent, the binding agent being selected from the group consisting of alginates, chitosan, water-insoluble polymers, plant oils having a melting point above room temperature, and combinations thereof.

20. The product of claim 19, wherein the product comprises an outer-water permeable pouch enclosing the agglomerated botanical particles.

21. The product of claim 19, further comprising an active ingredient selected from the group consisting of a tobacco material, a nicotine component, additional botanicals, nutraceuticals, stimulants, amino acids, vitamins, cannabinoids, cannabimimetics, terpenes, and combinations thereof.

22. The product of claim 21, wherein the active ingredient is absorbed in or adsorbed on the agglomerated botanical particles.

23. The product of claim 19, further comprising one or more additives selected from the group consisting of a flavoring agent, a salt, a sweetener, a filler, an additional binding agent, water, a humectant, an organic acid, a buffering agent, and combinations thereof.

24. The product of claim 23, wherein the one or more additives is absorbed in or adsorbed on the agglomerated botanical particles.

25. The product of claim 19, wherein the product has a water content in the range of about 20% to about 60% by weight, based on the total weight of the product.

26. The product of claim 19, wherein the product has a water content in the range of about 30% to about 50%, based on the total weight of the oral product.
